(19) United States
(12) Patent Application Publication
(10) Pub. No.: US 2023/0354979 A1
(43) Pub. Date: Nov. 9, 2023

(54) SCALP AND HAIR MANAGEMENT SYSTEM

(71) Applicant: Jungbin SONG, Seoul (KR)

(72) Inventor: Jungbin SONG, Seoul (KR)

(21) Appl. No.: 18/018,354

(22) PCT Filed: Jul. 6, 2021
(86) PCT No.: PCT/KR2021/008608

§ 371 Date: Jan. 27, 2023

(30) Foreign Application Priority Data
Jul. 6, 2020 (KR) 10-2020-0082834
Jul. 6, 2020 (KR) 10-2020-0082837
Jul. 6, 2020 (KR) 10-2020-0082840
Jul. 6, 2020 (KR) 10-2020-0082847

(51) Int. Cl. A45D 19/00; A45D 44/00; A61B 5/00; G16H 50/70; G16H 20/10; G06T 7/00

(52) U.S. Cl. CPC A45D 19/005; A45D 44/00; A61B 5/446; A61B 5/448; G16H 50/70; G16H 20/10; G06T 7/0014; A45D 2044/007; G16H 10/60

(57) ABSTRACT

A scalp and hair management system has increased precision by collecting user information, particularly decision-making information and electroencephalographic information so as to provide aspects of change in scalp and hair managing habits in order to apply a transtheoretical model relating to scalp and hair managing habits for users; provides, by means of realistic image information, changed state information in accordance with systemic management of scalp and hair; verifies effects of using multiple scalp and hair management programs in stages of changes of the scalp and hair management from an initial state; finds and provides a management program suitable for a user; analyzes and provides positive factors and negative factors of the management program; provides state information of the user in stages; and thus boosts motivation for management.

### BACKGROUND

The present disclosure relates to a scalp and hair management system, and more particularly, to a scalp and hair management system which: has increased precision by collecting user information, particularly decision-making information and electroencephalographic information so as to provide aspects of change in scalp and hair managing habits in order to apply a transtheoretical model relating to scalp and hair managing habits for users; provides, by means of realistic image information, changed state information in accordance with systemic management of scalp and hair; verifies effects of using multiple scalp and hair management programs in stages of changes of the scalp and hair management from an initial state; finds and provides a management program suitable for a user; analyzes and provides positive factors and negative factors of the management program; provides state information of the user in stages; and thus boosts motivation for management.

Various beauty subsidiary materials such as shampoo and rinse for scalp and hair management and beauty procedures are performed, but these acts are made by the intuitive judgment of an operator and are not systematically managed.

Accordingly, if big data information for verifying the effect using hair and scalp massage programs that correspond to the application of various beauty subsidiary materials and beauty procedures, and setting the period for parameters such as period of use in the change stage is collected and utilized, a person who has been managed by the change will be unlikely to return to the old habit.

In addition, in the case where the scalp and the hair are exposed to excessive stress and various chemicals or the sulfur or nitrogen compound from the exhaust gas of the car is attached to the scalp and hair, there is a very high possibility that the scalp and the hair will be physically or chemically damaged.

Sensitive and stimulated scalp environments increase the possibility of hair loss. In addition, damaged hair is difficult to recover. Therefore, the scalp and hair should be constantly managed in advance before damaged.

On the other hand, a transtheoretical model as an integrated theory model for explaining the changes in the behavior of individuals developed by Prochaska and Dictemente (1983) is a model for describing a spontaneous change of the individual behavior and is an integrated theory for describing a principle and a process of an action change regarding how the individual makes a new attempt for a health action and how the individual maintains the health action, and describes a human behavior change with a core of a change stage and a change process.

The present disclosure succeeds in developing a scalp and hair management system which: has increased precision by collecting user information, particularly decision-making information and electroencephalographic information so as to provide aspects of change in scalp and hair managing habits in order to apply a transtheoretical model as the number of users who sysmetically manage the scalp and hair by visiting a hair shop or a medical institution and apply a transtheoretical model relating to scalp and hair managing habits for users; provides, by means of realistic image information, changed state information in accordance with systemic management of scalp and hair; verifies effects of using multiple scalp and hair management programs in stages of changes of the scalp and hair management from an initial state; finds and provides a management program suitable for a user; analyzes and provides positive factors and negative factors of the management program; provides state information of the user in stages; and thus boosts motivation for management to complete the present disclosure.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present disclosure is contrived to solve the problem, and has been made in an effort to provide a scalp and hair management system for verifying an effect by using a plurality of scalp and hair management programs from an initial state for a change stage of a scalp and hair management action, and finding and providing a management program suitable for a user.

Further, the present disclosure has been made in an effort to provide a scalp and hair management system for boosting motivation for management by analyzing and providing positive factors and negative factors of the management program and providing state information for each step of a user.

Further, the present disclosure has been made in an effort to provide a scalp and hair management system for boosting motivation for management by analyzing and providing positive factors and negative factors of the management program and providing state information for each step of a user.

Further, the present disclosure has been made in an effort to provide a scalp and hair management system for activating scalp and hair management remotely for small groups or individuals by systematically analyzing and providing a change aspect of a scalp and hair management habit and good habit and bad habit information suitable for a user according to a change by applying a transtheoretical model for the scalp and hair management habit for users who wants to manage scalp and hair in a scalp or hair management room, a hair shop, a skin care room, etc.

In addition, the present disclosure has been made in an effort to provide a scalp and hair management system which is not only used as a faster and skilled expert training material through mobile apps, but also used for verifying the effect of the change of scalp and hair management acts using management apps such as hair and scalp massage.

Further, the present disclosure has been made in an effort to provide a scalp and hair management system for verifying effects of change stages of scalp and hair management acts by using management programs such as hair and scalp massages and enabling a definite period for a program utilization period in a change stage during a predetermined period, and showing that a probability that a person receiving change stage management will return to old habits is lower and more positive than a probability that persons in a behavior stage will return to the old habits.

Further, the present disclosure has been made in an effort to provide a scalp and hair management system which is to be applied even to a home care scalp scaling management program by identifying that a health condition of a scalp is improved, and to be applied even to an individual customized scalp and hair management program.

An exemplary embodiment of the present disclosure provides a scalp and hair management system including: a hair and scalp management terminal 100a; a network; and a hair diagnoser group 100bg constituted by a plurality of hair diagnosers 100b, in which the hair and scalp management terminal 100a includes
a member joining module 131 which generates initial hair/scalp analysis information by comparing the high-magnification photographing image with the pattern information of the normal state, the dry state, the oily state, the sensitive state, the dandruff scalp, and the hair loss state stored in the big data server 600, and then stores, in the storage unit 140, the initial hair/scalp analysis information jointly with the hair/scalp state information with the user ID as the metadata in the user unit information, and provides the mobile diagnoser 100b with the initial hair/scalp analysis information and outputs the provided initial hair/scalp analysis information onto the hair diagnoser 100b,
stores patterns distributed and stored in the DCS DB for each state by a distribution file program and the big data server 600, stores, in at least one DCS DB, a plurality of pattern own information for each state or information on each state pattern which is inclined at a predetermined angle, and information on each state pattern which is inversely inclined, and extracts state category of the DCS DB matching by comparing the information of the DCS DB and the high-magnification shooting image as the initial hair/scalp analysis information, and
extracts characteristics of a pixel from features of the pixel for hair and scalp regions, i.e., brightness of the pixel, a color of the pixel, a line formed by the pixel, and a form of the pixel which is changed according to the elapse of the time, in addition to the form, and determines a pixel corresponding to a state pattern including a characteristic similar to the characteristic of each pixel as a similar range,
a behavior change analysis module 132 which extracts hair and scalp management behavior information according to the initial hair/scalp analysis information analyzed by the member joining module 131 from the storage unit 140, and then provide the hair and scalp management behavior information and the user ID to the hair diagnoser 100b through the network, and outputs the hair and scalp management behavior information onto the hair diagnoser 100b and provides the hair and scalp management behavior information including normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the normal state, the dry state, the oily state, the sensitive state, dandruff scalp, and hair loss state,
controls the transceiving unit 110 to receive decision making information of the user from the hair diagnoser 100b through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then stores the decision making information in user unit information jointly by using a user ID as metadata,
collects big data for clear period setting for a us period of hair and scalp management behavior information by a method of collecting the decision making information of the mobile diagnoser 300 in the change step during the predetermined period in order to verify an effect by using a hair and scalp massage program providing the change step of the hair and scalp management behavior as the hair and scalp management behavior information depending on the initial hair/scalp analysis information, and
collects the decision making information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and receives information collected through the input unit of the mobile diagnoser 300 when collecting the information, and
a relevance analysis module 133 which analyzes relevance between the hair and scalp management behavior information which is the hair and scalp management behavior of the user, and each parameter information of the decision making information for the hair and scalp management behavior information which is a behavior change process characteristic, and
extracts, from the big data server 600, change parameter information (including change parameter information for a shape and a color) individually set according to the hair and the scalp when changing each hair/scalp state to the normal state for each predetermined change step (a natural number of 2 or more) to extract a predetermined change step through a comparison with a high-magnification photographing image which is image information photographed toward the scalp inside the hair received from the hair diagnoser 100b through the network between conduction depending on each change parameter information and each parameter information of one decision making information and conduction of parameter information of another decision making information.

In this case, the member subscription module 131 stores an ID, a password, and personal information of the user when the ID, the password, and the personal information of the user are input through the input/output unit 110, and controls the transceiving unit 120 to perform log-in through the user ID and password through the network by one hair diagnoser 100b constituting the mobile diagnoser group 100bg, and then controls the transceiving unit 120 to receive a high-magnification photographing image of the user from the hair diagnoser 100b as hair/scalp state information, and stores the high-magnification photographing image with the user ID as metadata in the user unit information.

Another exemplary embodiment of the present disclosure provides a scalp and hair management system 1 having a structure in which a mobile terminal group 100g constituted by one or more mobile terminals 100 is connected to the hair diagnoser 100b formed for each beauty salon through short-range wireless communication, and each mobile terminal 100 is connected to a scalp and hair management server 400, in which the scalp and hair management server 400 includes
a first information collection module 421 which controls the transceiving unit 410 to receive a diagnoser ID of at least one hair diagnoser 100b of which data session is connected to the mobile terminal 100 by short-range wireless communication jointly with a first terminal identification number (IMEI) of the mobile terminal 100 according to an access from one mobile terminal 100 through the network, and then stores the diagnoser ID of at least one hair diagnoser 100b in a database 430 by using the first terminal identification number (IMEI) of the mobile terminal 100 as metadata, and specifies the first terminal identification number (IMEI) stored as the metadata as a manager number,
a second information collection module 422 which control the transceiving unit 410 to receive at least one diagnoser ID allocated to another mobile terminal 100 having a second terminal identification number jointly with the second terminal identification number of at least one other mobile terminal 100 for forming the same mobile terminal group 100g from the mobile terminal 100 specified as the manager number through the network, and then specify the first terminal identification number as a group name, and store both each second terminal identification number and the allocated diagnoser ID as lower category information of a specified group name, wherein one mobile terminal 100 corresponding to the first terminal identification number may be operated by a manager who professionally manages the scalp and the hair, such as the beauty salon, a hair shop, etc., and the other mobile terminal 100 corresponding to the second terminal identification number may be operated by a user who receives management for the scalp and the hair by the manager, and the diagnoser ID which one mobile terminal 100 corresponding to the first terminal identification number registers in the database 430 may be IDs of all hair diagnosers 100b provided at a place operated by the manager, and the diagnoser ID which the other mobile terminal 100 corresponding to the second terminal identification number registers may be an ID of the mobile diagnoser 100b specified by the manager or the user among all hair diagnosers 100b provided at the place operated by the manager, and
stores, when an ID, a password, and personal information of the user are input through each mobile terminal 100, the diagnoser ID of the hair diagnoser 100b specified to the user as a lower category of each terminal identification information on the database 430 as “user unit information” at the time of receiving the ID, the password, and the personal information of the user through the network, and
a scalp and hair analysis module 423 which controls the transceiving unit 410 to perform log-in through the user ID and the password through the network by each mobile terminal 100, and then controls the transceiving unit 410 to transmit the diagnoser ID specified in the user unit information corresponding to the user ID to the mobile terminal 100 which is logged in through the network,
controls the transceiving unit 410 to receive the high-magnification photographing image of the user provided from the hair diagnoser 100b as hair/scalp state information from the logged-in mobile terminal 100 in the state of connecting the data session with the hair diagnoser 100b corresponding to the specified diagnoser ID through the short-range wireless communication, and stores the high-magnification photographing image as the lower category of the user ID in the user unit information,
provides, when there is a plurality of diagnoser IDs specified to one user, diagnoser ID information which is not used through the query for whether to use the mobile diagnoser 100b corresponding to each diagnoser ID to the mobile terminal 100 operated by the manager corresponding to the first terminal identification number to each logged-in mobile terminal 200 through the network, wherein when the mobile terminal 100 operated by the manager automatically acquires driving state information through a management app by a short-range communication method for each hair diagnoser 100b, receives the acquired driving state information from the mobile terminal 100,
generates initial hair/scalp analysis information by comparing the high-magnification shooting image with the pattern information of the normal state, the dry state, the oily state, the sensitive state, the dandruff scalp, and the hair loss state stored in the big data server 600, and then stores, in the database 430, the initial hair/scalp analysis information jointly with the hair/scalp state information with the user ID as the metadata in the user unit information, and controls the transceiving unit 410 to transmit the initial hair/scalp analysis information to each logged-in mobile terminal 100 to store and output the transmitted initial hair/scalp analysis information onto each logged-in mobile terminal 100,
receives, from the hair diagnoser 100b, the high-magnification shooting image which is image information acquired by photographing the scalp in the hair of the user shot by a high-magnification camera formed in the logged-in mobile terminal 100 as the hair/scalp state information, and then controls the transceiving unit 410 to deliver the hair/scalp state information to the AI server 500 through the network 500 to control the transceiving unit 410 to be returned with the generated initial hair/scalp analysis information through the analysis through collection data distributively stored in the DCS DB by the distribution file program based on the big data on the AI server 500 is generated,
extracts, from the database 430, hair and scalp management behavior information depending on the analyzed initial hair/scalp analysis information, and then controls the transceiving unit 410 to transmit the hair and scalp management behavior information and the user ID to the logged-in mobile terminal 100 through the network and output the hair and scalp management behavior information and the user ID onto the logged-in mobile terminal 100,
controls the transceiving unit 410 to receive decision making information of the user from each mobile terminal 100 corresponding to the second terminal identification number through the network for each predetermined cycle within a predetermined period when conducting the behavior change analysis during a predetermined period in order to determine a behavior change process depending on a change step depending on hair and scalp management behavior information conduction depending on initial hair/scalp analysis information, and then stores the decision making information in user unit information jointly by using a user ID as metadata,
collects big data for clear period setting for a us period of hair and scalp management behavior information by a method of collecting the decision making information from each mobile terminal 100 corresponding to the second terminal identification number in the change step during the predetermined period in order to verify an effect by using a hair and scalp massage program providing the change step of the hair and scalp management behavior as the hair and scalp management behavior information depending on the initial hair/scalp analysis information,
collects the decisional information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and collects an intention declaration representing whether to use the parameter information through a touch screen which is the input unit of each mobile terminal 100 corresponding to the second terminal identification number,
analyzes relevance between the hair and scalp management behavior information which is the hair and scalp management behavior of the user, and each parameter information of the decision making information for the hair and scalp management behavior information which is a behavior change process characteristic though a request for the AI server 500,
in a case where the predetermined change step information is one of whether to maintain the normal state and whether each state is changed to the normal state is positive from a time before the hair and scalp management behavior and is matched with at least one of the decision making of the positive aspect (pros) and the decision making of the negative aspect (cons) opposite thereto in each parameter information of the decision making information for the hair and scalp management behavior information, analyzes the case as “the positive relevance information” to store positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state),
on the contrary, in a case where the predetermined change step information is not one of whether to maintain the normal state and whether each state is changed to the normal state is positive and is matched with at least one of the decision making of the positive aspect (pros) and the decision making of the negative aspect (cons) opposite thereto in each parameter information of the decision making information for the hair and scalp management behavior information, analyzes the case as “the negative relevance information” to store positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state),
analyzes relevance between the hair and scalp management characteristic of the user and a transtheoretical model configuration factor, and collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected by the database 430 for each user for each predetermined cycle to analyze a step indicating an intention for a behavior change of the transtheoretical model, and controls the transceiving unit 410 to generate degree information for a change from a current hair and scalp state of the user to the normal state and generates the generated degree information as change process information, additionally receive decision making information and self efficacy information through the input unit of the mobile terminal 100 for the user and generate step information for one of pre-plan, plan, preparation, behavior, and maintenance as a step showing an intention regarding the behavior change of the practice performer and transmit the generated step information to the mobile terminal 100 through the network to control to output the degree information and the step information to the output unit of the mobile diagnoser 300,
wherein in the decisional balance, two elements of the decision making, i.e., pros and cons may be configured in the transtheoretical model, the decisional balance may mean comparing and evaluating the pros and the cons generated to the user when the user changes any behavior, and may be used as a dependent variable or an intermediary variable as a measure to confirm that the behavior change occurs and the change step is progressed,
it is assumed that since the decision making is determined according to a degree of relative importance of the individual, a regular practice behavior is attempted or is not continued until a recognition level (pros) for the positive aspect which the regular practice gives exceeds a recognition level (cons) for the negative aspect in relation to the regular practice behavior, and provided as a result value calculated through a multiplication for a quantitative numerical value for each of approval and non-approval items according to an input through the input unit of the mobile terminal 100 for the approval and the non-approval for each component information of the hair and scalp management behavior information and a predetermined weight value,
in the self efficacy, a multiplication of the weight value which is in proportion to a percentage of the change process information generated when the change step positively increases and a predetermined positive quantitative numerical value corresponding to each approval for the hair and the scalp of the user, and a result value of aggregating multiplied values may be generated, and a result value through multiplication for a predetermined negative quantitative numerical value corresponding to overall denial for the hair and the scalp of the user according to the weight which is in proportion to the percentage of the change process information generated when the change step negatively increases may be generated, and
aggregates quantitative numerical values of the change process information, the decisional balance information, and the self efficacy information, and then generates step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and stores the step information in the database 430.

In this case, the hair and scalp management behavior information includes each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the normal state, the dry state, the oily state, the sensitive state, dandruff scalp, and hair loss state.

In this case, scalp and hair management server 400 further includes a scalp and hair management module 425 which controls the transceiving unit 410 to extract component information which is most frequently generated among the positive component information of the positive relevance and the negative component information of the negative relevance during moving to a higher step in information on first steps of pre-plan, plan, preparation, behavior, and maintenance as the change steps presented in the theoretical model and transmit the component information to the mobile terminal 100 through the network as recommended behavior information most suitable for the user to provide the most suitable recommended behavior information for continuation to a next step and manage the user.

Yet another exemplary embodiment of the present disclosure provides scalp and hair management system including a mobile terminal 100, a hair diagnoser group 100bg constituted by a plurality of hair diagnosers 100b, a network, and a scalp and hair management server 400, in which the scalp and hair management server 400 includes
a transtheoretical model providing module 422, and
a related factor analysis module 423 which provides approval item information extracted through extraction to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the transtheoretical model providing module 422 to know that a positive mind of the user progresses the movement to the higher step, when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the decisional balance information to provide the decisional balance information in order to determine first to fifth range steps of five steps of pre-plan, plan, preparation, behavior, and maintenance as the change steps presented in the transtheoretical model according to user information analysis.

In this case, when the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the decisional balance in order to determine the first to fifth range steps to provide the decisional balance information, approval item information extracted through extraction is provided to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the transtheoretical model providing module 422 to know that a negative mind of the user progresses the movement to the lower step.

Further, when the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, approval item information extracted through extraction is provided to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the transtheoretical model providing module 422 to know that a positive mind of the user causes the movement to the higher step.

Further, when the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, non-approval item information extracted through extraction is provided to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the transtheoretical model providing module 422 to know that a negative mind of the user causes the movement to the lower step.

Further, the scalp and hair management server 400 further includes a management conduction module 424 which extracts, as first information, positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state) analyzed as the “positive relevance” stored in the database 430 for each user ID.

Further, the management conduction module 324 extracts, as second information, positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state) analyzed as the “negative relevance” stored in the database 430.

Further, the management conduction module 324 generates graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal 100 is represented as a timeline between neighboring steps.

Further, the management conduction module 324 provides approval item information (first information) extracted through extraction with respect to response to the positive aspect in the decisional balance information and the approval item for forming the decisional balance information during moving to the higher step in information on five steps of pre-plan, plan, preparation, behavior, and maintenance as the change steps presented in the theoretical model presented by the related factor analysis module 423 and approval item information (second information) extracted through extraction with respect to response to the negative aspect in the decisional balance information and the approval items for forming the decisional balance information during moving to the lower step in information on five steps of pre-plan, plan, preparation, behavior, and maintenance as the change steps presented in the theoretical model to the mobile terminal 100 through the network for each user ID in real time.

Further, the transtheoretical model providing module 422 receives the initial hair/scalp analysis information from the AI server 500 through an initial hair/scalp analysis request for the AI server 500 through the network for the high-magnification photographing image included in the hair and scalp state information (hereinafter, referred to hair/scalp state information) of the user provided from the hair diagnoser 100b.

Further, the transtheoretical model providing module 422 extracts hair and scalp management behavior information according to the analyzed initial hair/scalp analysis information from the database 430, and then provide the hair and scalp management behavior information and the user ID to the mobile terminal 100 through the network, and outputs the hair and scalp management behavior information onto the mobile terminal 100 and provides the hair and scalp management behavior information including normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (including the number of times of massage and the cycle of the message according to the state, etc.) for each analyzed initial hair/scalp analysis information corresponding to the normal state, the dry state, the oily state, the sensitive state, dandruff scalp, and hair loss state.

Further, the transtheoretical model providing module 422 controls the transceiving unit 410 to receive decision making information of the user from the hair diagnoser 100b through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then stores the decision making information in user unit information jointly by using a user ID as metadata.

Further, the transtheoretical model providing module 422 collects the decision making information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information,
analyzes relevance between the hair and scalp management behavior information which is the hair and scalp management behavior of the user, and each parameter information of the decision making information for the hair and scalp management behavior information which is a behavior change process characteristic though a request for the AI server 500,
in a case where one of whether to maintain the normal state and whether each state is changed to the normal state received from the AI server 500 is positive and the decision making of the positive aspect (pros) and the decision making of the negative aspect (cons) opposite thereto are matched with each other in each parameter information of the decision making information for the hair and scalp management behavior information, the relevance analysis module 500 analyzes the case as positive relevance to component information of the hair and scalp management behavior information jointly with the user unit information as the positive relevance information,
in a case where both whether to maintain the normal state and whether each state is changed to the normal state is negative and the decision making of the positive aspect (pros) and the decision making of the negative aspect (cons) opposite thereto are matched with each other in each parameter information of the decision making information for the hair and scalp management behavior information, analyzes the case as negative relevance to store component information of the hair and scalp management behavior information jointly with the user unit information as the negative relevance information, and
analyzes relevance with hair and scalp management characteristics of the user and a transtheoretical model configuration factor.

Still yet another exemplary embodiment of the present disclosure provides a scalp and hair management system 1 comprising a user personal terminal group constituted by a plurality of user personal terminals 10, a network, a scalp and hair management server 400, an AI server 500, and a big data server 600, wherein each user personal terminal 10 includes a mobile terminal 100, a hair diagnoser 100b, and an HMD 110b, and each of a diagnoser ID and an HMD ID which one mobile terminal 100 corresponding to a terminal identification number registers in a database 430 performs short-range wireless communication with the mobile terminal 100 with a personal smart device operated by a user, in which the scalp and hair management server 400 includes
an information collection module 421 which controls the transceiving unit 410 to receive a diagnoser ID of at least one hair diagnoser 100b and an HMD ID of the HMD 100a of which data session is connected to the mobile terminal 100 and the HMD 100a from one mobile terminal 100 jointly with the terminal identification number (IMEI) of the mobile terminal 100 according to an access through the network, and then stores the diagnoser ID and the HMD ID by using the terminal identification number (IMEI) of the mobile terminal 100 as metadata in the database 430, controls the transceiving unit 410 to store each terminal identification number as the metadata by using the user ID, the password, the diagnoser ID, and the HMD ID on the database 430 as the “user unit information” at the time of receiving the ID and the password of the user through the network and perform log-in through the user ID and the password through the network by the mobile terminal 100 when an ID and a password of the user are input through each mobile terminal 100, controls the transceiving unit 410 to receive the high-magnification photographing image of the user from the scalp and hair diagnoser 100 connected to the mobile terminal 100 by short-range wireless communication from the mobile terminal 100 as hair and scalp state information (hereinafter, referred to as hair/scalp state information) and controls the transceiving unit 410 to receive brainwave information from a brainwave measurement device connected to the mobile terminal 100 by the short-range wireless communication, and stores the high-magnification photographing image (hair/scalp state information) and initial reference brainwave information by using the user ID as the metadata in the user unit information,
an initial information analysis module 422 which receives the initial hair/scalp analysis information from the AI server 500 through an initial hair/scalp analysis request for the AI server 500 through the network for the high-magnification photographing image,
receives, from the mobile terminal 100, brainwave change information from a brainwave measurer connected to the mobile terminal 100 by the short-range wireless communication at the time of outputting the initial hair/scalp analysis information to the output unit of the mobile terminal 100 according to the transmission of the initial hair/scalp analysis information to the mobile terminal 100, and stores each terminal identification number as the metadata on the database 430 with first change brainwave information as the “user unit information”,
extracts hair and scalp management behavior information according to the analyzed initial hair/scalp analysis information from the database 430, and then provide the hair and scalp management behavior information and the user ID to the mobile terminal 100 through the network, and outputs the hair and scalp management behavior information onto the mobile terminal 100 and provides the hair and scalp management behavior information including normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each analyzed initial hair/scalp analysis information corresponding to the normal state, the dry state, the oily state, the sensitive state, dandruff scalp, and hair loss state,
controls the transceiving unit 410 to receive decision making information of the user from the scalp and hair diagnoser 100 through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then stores the decision making information in user unit information jointly by using a user ID as metadata,
controls the transceiving unit 410 to receive the high-magnification photographing image (hair/scalp state information) collected by the hair diagnoser 100b from the mobile terminal 100 through the network for each predetermined cycle within a predetermined period in order to collect the decision making information, and then provides the high-magnification photographing image (hair/scalp state information) jointly with received intermediate analysis result information for each cycle to generate the decision making information as provided reaction information (response and brainwave information to O and X through the input unit) according to each query,
a transtheoretical model providing module 423 which collects, from the mobile terminal connected to a brainwave measurement device, brainwave change information according to a first query for decision making of the positive aspect (pros) according to the use of each recommended shampoo information/decision making of the negative aspect (cons) opposite thereto, brainwave change information according to a second query for decision making of the positive aspect (pros) according to the use of each scalp scaling information/decision making of the negative aspect (cons) opposite thereto, and brainwave change information according to a third query for decision making of the positive aspect (pros) according to the use of scalp massage information/decision making of the negative aspect (cons) opposite thereto, as brainwave information,
collects the decision making information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information,
collects the first to third parameter information by a scheme of providing the brainwave change information measured by the brainwave measurement device to the mobile terminal 100 as reference information, and receiving whether to use the component constituting each hair and scalp management behavior information from the mobile terminal 100 for an intention expression (O and X) of the user through the input unit of the mobile terminal 100,
analyzes the brainwave change information provided to the mobile terminal 100 by the transtheoretical model providing module 423 by decision making of a negative aspect (cons) opposite thereto when a predetermined frequency or more increases in each initial reference brainwave information and a positive aspect (pros) when the predetermined frequency or more does not increase for a user who stares at the intermediate analysis result information for each cycle while the initial reference brainwave information is primarily classified into a delta wave, a theta wave, an alpha wave, a beta wave, and an gamma wave,
analyzes whether to maintain each normal state, whether the dry state is changed to the normal state, whether the oily state is changed to the normal state, whether the sensitive state is changed to the normal state, whether the dandruff scalp is changed to the normal state, and whether the hair loss state is changed to the normal state corresponding to the hair and scalp management behavior information, and the initial hair/scalp analysis information depending on each parameter information of the decision making information,
in a case where one of whether to maintain the normal state and whether each state is changed to the normal state received from the AI server 500 is positive and the decision making of the positive aspect (pros) and the decision making of the negative aspect (cons) opposite thereto are matched with each other in each parameter information of the decision making information for the hair and scalp management behavior information, analyzes the case as positive relevance to component information of the hair and scalp management behavior information jointly with the user unit information as the positive relevance information,
in a case where both whether to maintain the normal state and whether each state is changed to the normal state is negative and the decision making of the positive aspect (pros) and the decision making of the negative aspect (cons) opposite thereto are matched with each other in each parameter information of the decision making information for the hair and scalp management behavior information, analyzes the case as negative relevance to store component information of the hair and scalp management behavior information jointly with the user unit information as the negative relevance information,
analyzes relevance with hair and scalp management characteristics of the user and a transtheoretical model configuration factor,
collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected on the database 430 for each user for each predetermined cycle to analyze a step indicating an intention for a behavior change of the transtheoretical model,
controls the transceiving unit 410 to generate degree information for a change from a current hair and scalp state of the user to the normal state and generates the generated degree information as change process information, additionally receive decision making information and self efficacy information through the input unit of the mobile terminal 100 for the user and generate step information for one of pre-plan, plan, preparation, behavior, and maintenance as a step showing an intention regarding the behavior change of the practice performer and transmit the generated step information to the mobile terminal 100 through the network to control to output the degree information and the step information to the output unit of the mobile terminal 100, and controls the transceiving unit 410 to aggregate quantitative numerical values of the change process information, the decisional balance information, and the self efficacy information, and then generate step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and transmit the step information to the mobile terminal 100 to control the step information to the output unit of the mobile terminal 100, and
a feedback providing module 425 which generates a VR image according to the timeline for a 2D image for the high-magnification photographing image (hair/scalp state information) for generating the intermediate analysis result information for each cycle from an initially collected high-magnification photographing image (hair/scalp state information), and provides the generated VR image to the mobile terminal 100 through the network,
in order to generate the VR image according to the cycle of each timeline, determines a plurality of focal positions and focuses for the high-magnification photographing image which is the 2D image, and computes a focal distance between respective focal positions for a plurality of focus 2D image data and computes a depth value which is in inverse proportion to the focal distance computed between the respective focal positions when a plurality of multiple focus 2D image data corresponding to the plurality of focus positions and focuses determined,
performs decoding of extracting and informationizing image information (a color, chroma, and brightness) corresponding to each pixel for a range of image data corresponding to each focus number of the high-magnification photographing image (hair/scalp state information) to generate decoded data and generates a polygon set to which a depth value computed for each pixel is reflected for a polygon which is a basic unit for expressing the decoded data as a 3D shape and then performs pixel mapping of attaching the decoded data onto the polygon set to generate each specified focus-specific virtual reality (VR) image, and
provides the set of the VR image according to the cycle of each timeline to the mobile terminal 100 through the network to provide the scalp and hair state according to the hair and scalp management behavior information which is a behavior change process as the time elapses to the user as a realistic VR image through the HMD 100a connected to the mobile terminal 100 through the short-range wireless communication.

In this case, the scalp and hair management system further includes an information providing module 424 providing, when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the decisional balance information to provide the decisional balance information in order to determine first to fifth range steps, approval item information extracted through extraction to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the initial information analysis module 422 to know that a positive mind of the user causes the movement to the higher step,
providing, when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the decisional balance information to provide the decisional balance information in order to determine first to fifth range steps, non-approval item information extracted through extraction to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the initial information analysis module 422 to know that a negative mind of the user causes the movement to the lower step,
when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, approval item information extracted through extraction is provided to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the initial information analysis module 422 to know that a positive mind of the user progresses the movement to the higher step,
when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, providing non-approval item information extracted through extraction to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the initial information analysis module 422 to know that a positive mind of the user causes the movement to the lower step,
extracting, as first information, positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state) analyzed as the “positive relevance” stored in the database 430 for each user ID,
extracting, as second information, positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state) analyzed as the “negative relevance” stored in the database 430, and
generating graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal 100 is represented as a timeline between neighboring steps, and providing approval item information (first information) extracted through extraction with respect to response to the positive aspect in the decisional balance information and the approval item for forming the decisional balance information during moving to the higher step in information on five steps of pre-plan, plan, preparation, behavior, and maintenance as the change steps presented in the theoretical model presented by the information providing module 424 and approval item information (second information) extracted through extraction with respect to response to the negative aspect in the decisional balance information and the approval items for forming the decisional balance information during moving to the lower step in information on five steps of pre-plan, plan, preparation, behavior, and maintenance as the change steps presented in the theoretical model to the mobile terminal 100 through the network for each user ID in real time.

According to an exemplary embodiment of the present disclosure, a scalp and hair management system can verify an effect by using a plurality of scalp and hair management programs from an initial state for a change phase of a scalp and hair management action, and find and provide a management program suitable for to a user.

Further, according to another exemplary embodiment of the present disclosure, a scalp and hair management system can boosting motivation for management by analyzing and providing positive factors and negative factors of the management program and providing state information for each step of a user.

Further, according to yet another exemplary embodiment of the present disclosure, a scalp and hair management system can boosting motivation for management by analyzing and providing positive factors and negative factors of the management program and providing state information for each step of a user.

Further, according to still yet another exemplary embodiment of the present disclosure, a scalp and hair management system can activate scalp and hair management remotely for small groups or individuals by systematically analyzing and providing a change aspect of a scalp and hair management habit and good habit and bad habit information customized to a user according to a change by applying a transtheoretical model for the scalp and hair management habit for users who wants to manage scalp and hair in a scalp or hair management room, a hair shop, a skin care room, etc.

Further, according to still yet another exemplary embodiment of the present disclosure, a scalp and hair management system can be not only used as a faster and skilled expert training material through mobile apps, but also verify the effect of the change of scalp and hair management acts using management apps such as hair and scalp massage.

Further, according to still yet another exemplary embodiment of the present disclosure, a scalp and hair management system can verify effects of change stages of scalp and hair management acts by using management programs such as hair and scalp massages and enable a definite period for a program utilization period in a change stage during a predetermined period, and show that a probability that a person receiving change stage management will return to old habits is lower and more positive than a probability that persons in a behavior stage will return to the old habits.

Further, according to still yet another exemplary embodiment of the present disclosure, a scalp and hair management system is to be applied even to a home care scalp scaling management program by identifying that a health condition of a scalp is improved, and to be applied even to an individual customized scalp and hair management program.

The effects of the present disclosure are not limited to the aforementioned effect, and other effects, which are not mentioned above, will be apparent to a person having ordinary skill in the art from the description of the claims.

### BRIEF DESCRIPTION OF THE DRAWINGS

FIG. 1 is a diagram illustrating a scalp and hair management system 1 according to an exemplary embodiment of the present disclosure.

FIG. 2 is a block diagram illustrating a component of a hair and scalp management terminal 100a in the scalp and hair management system 1 according to the exemplary embodiment of the present disclosure.

FIG. 3 is a diagram illustrating a hair diagnoser 100b used in the scalp and hair management system 1 according to the exemplary embodiment of the present disclosure.

FIG. 4 is a diagram illustrating a scalp and hair management system 1 according to another exemplary embodiment of the present disclosure.

FIG. 5 is a block diagram illustrating a component of a hair and scalp management server 400 in the scalp and hair management system 1 according to the exemplary embodiment of the present disclosure.

FIG. 6 is a diagram illustrating a concept of mobile based remote scalp and hair management performed by the scalp and hair management server 400 in the scalp and hair management system 1 according to the exemplary embodiment of the present disclosure.

FIG. 7 is a diagram illustrating a scalp and hair management system 1 according to another exemplary embodiment of the present disclosure.

FIG. 8 is a block diagram illustrating a component of a hair and scalp management server 400 in the scalp and hair management system 1 according to another exemplary embodiment of the present disclosure.

FIG. 9 is a diagram illustrating a scalp and hair management system 1 according to yet another exemplary embodiment of the present disclosure.

FIG. 10 is a block diagram illustrating a component of a hair and scalp management server 400 in the scalp and hair management system 1 according to still yet another exemplary embodiment of the present disclosure.

FIG. 11 is a diagram for describing pattern information of a normal state (FIG. 1A), a dry state (FIG. 11B), an oily state (FIG. 11C), a sensitive state (FIG. 11), a dandruff scalp (FIG. 11E), and a hair loss state (FIG. 11F) by an AI server 500 in the scalp and hair management system 1 according to still yet another exemplary embodiment of the present disclosure.

FIGS. 12 to 14 are diagrams for describing each stage information of one recommended shampoo information (FIG. 12) in hair and scalp management action information provided by the scalp and hair management server 400 (FIG. 12), each stage information included in one scalp scaling information in the hair and scalp management action information (FIG. 12), each stage information included in one scalp scaling information in the hair and scalp management action information (FIG. 13), and one scalp massage information in the hair and scalp management action information (FIG. 14) in the scalp and hair management system 1 according to still yet another exemplary embodiment of the present disclosure.

FIGS. 15 to 17 are diagrams for describing information collection of collecting information by the scalp and hair management server 400 in the scalp and hair management system 1 according to still another exemplary embodiment of the present disclosure.

### DETAILED DESCRIPTION OF THE INVENTION

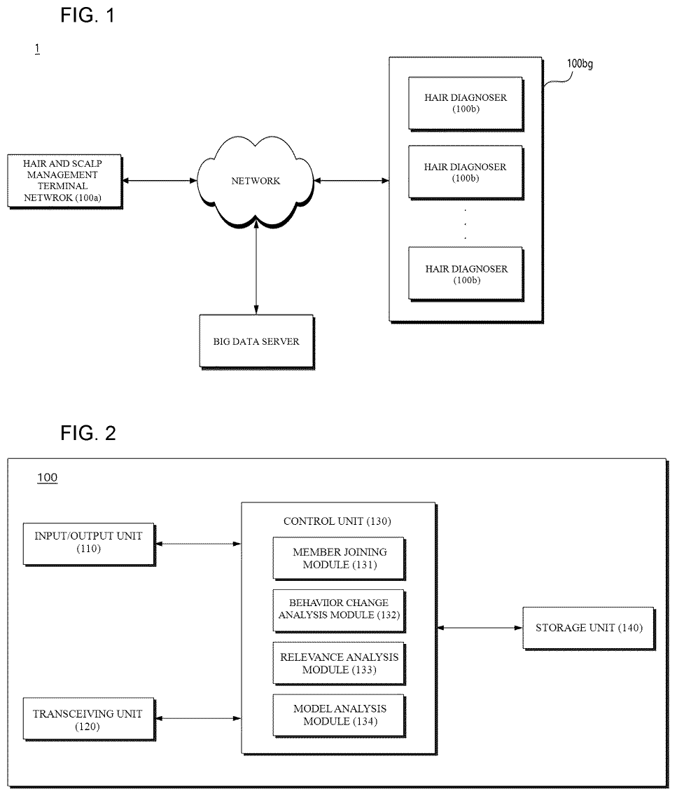

FIG. 1 is a diagram illustrating a scalp and hair management system 1 according to an exemplary embodiment of the present disclosure. Referring to FIG. 1, the scalp and hair management system 1 may include a hair and scalp management terminal 100a, a network, a hair diagnoser group 100bg constituted by a plurality of hair diagnosers 100b, and a big data server 600.

The network is a communication network which is a high-speed backbone network of a large communication network capable of a large-capacity, long-distance voice and data service, and may be a next-generation wired and wireless network for providing a high-speed multimedia service. When the network is a mobile communication network, the network may be a synchronous mobile communication network or an asynchronous mobile communication network. As an exemplary embodiment of the asynchronous mobile communication network, a wideband code division multiple access (WCDMA) type communication network may be used. In this case, although not illustrated in the figure, the network may include a radio network controller (RNC). Meanwhile, the WCDMA network is described as an example, but the network may be a 3G LTE network, a 4G network, a next-generation communication network such as other 5G, and an IP network based on other IPs. The network serves to mutually transfer signals and data among the hair and scalp management terminal 100a, the network, the hair diagnoser group 100bg constituted by the plurality of hair diagnosers 100b, the big data server 600, and other systems.

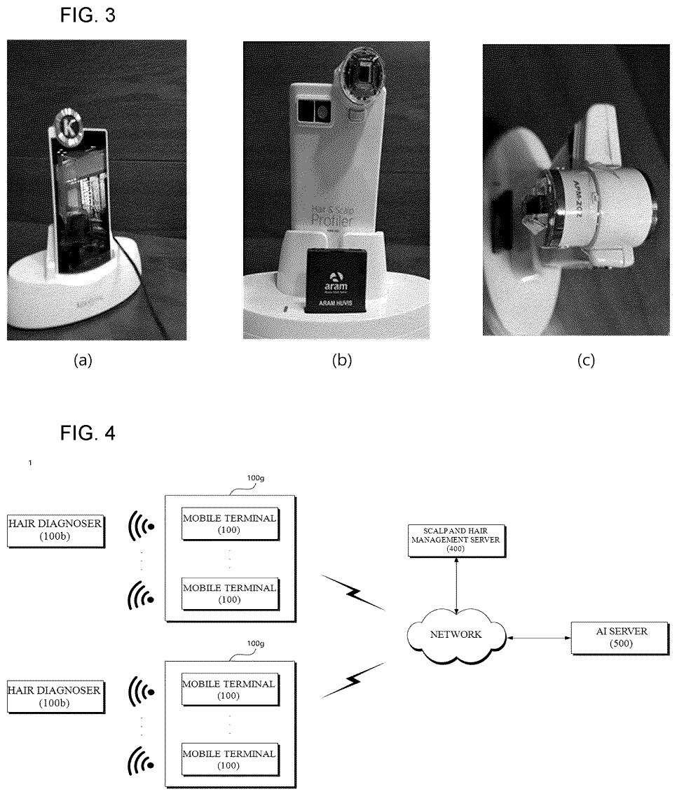

FIG. 2 is a block diagram illustrating a component of a hair and scalp management terminal 100a in the scalp and hair management system 1 according to an exemplary embodiment of the present disclosure. FIG. 3 is a diagram illustrating a hair diagnosis apparatus 100b used in the scalp and hair management system 1 according to an exemplary embodiment of the present disclosure.

First, referring to FIG. 2, the hair and scalp management terminal 100a may include an input/output unit 110, a transceiving unit 120, a control unit 130, and a storage unit 140. The control unit 130 may include a member joining module 131, a behavior change analysis module 132, a relevancy analysis module 133, and a model analysis module 134.

When an ID, a password, and personal information of a user are input through the input/output unit 110, the member joining module 131 may store the ID, the password, and the personal information of the user in the storage unit 140 as user unit information.

Thereafter, the member joining module 131 may control the transceiving unit 120 to perform log-in through the user ID and password through the network by one hair diagnoser 100b constituting the mobile diagnoser group 100bg, and then control the transceiving unit 120 to receive a high-magnification photographing image of the user from the hair diagnoser 100b as hair/scalp state information, and store the high-magnification photographing image with the user ID as metadata in the user unit information. To this end, the user preferably performs the log-in the user ID and the password thereof in an input unit which may be formed as a touch screen for one of the mobile diagnosers 100b of the type illustrated in FIG. 3, which is installed in each region.

Thereafter, the member joining module 131 may generate initial hair/scalp analysis information by comparing the high-magnification photographing image with the pattern information of the normal state, the dry state, the oily state, the sensitive state, the dandruff scalp, and the hair loss state stored in the big data server 600, and then store, in the storage unit 140, the initial hair/scalp analysis information jointly with the hair/scalp state information with the user ID as the metadata in the user unit information, and provide the mobile diagnoser 100b with the initial hair/scalp analysis information and output the provided initial hair/scalp analysis information onto the hair diagnoser 100b.

More specifically, the member joining module 131 may receive, from the hair diagnoser 100b, the high-magnification photographing image which is image information acquired by shooting the scalp in the hair of the user shot by a high-magnification camera formed in the hair diagnoser 100b as the hair/scalp state information, and then generate the initial hair/scalp analysis information through analysis of collected data distributed and stored in a DCS DB and a machine learning algorithm by the hair/scalp state information and a distributed file program on the big data server 600.

Here, the member joining module 131 stores patterns distributed and stored in the DCS DB for each state by a distribution file program on the high-magnification photographing image and the big data server 600, stores, in at least one DCS DB, a plurality of pattern own information for each state or information on each state pattern which is inclined at a predetermined angle, and information on each state pattern which is inversely inclined, and as a result, state category of the DCS DB matching by comparing the information of the DCS DB and the high-magnification photographing image may be extracted as the initial hair/scalp analysis information.

The member joining module 131 may extract characteristics of a pixel from features of the pixel for hair and scalp regions, i.e., brightness of the pixel, a color of the pixel, a line formed by the pixel, and a form of the pixel which is changed according to the elapse of the time, in addition to the form, and determine a pixel corresponding to a state pattern including a characteristic similar to the characteristic of each pixel as a similar range.

The behavior change analysis module 132 may extract, from the storage unit 140, hair and scalp management behavior information depending on initial hair/scalp analysis information analyzed by the member joining module 131, and then provide the hair diagnoser 100b with the hair and scalp management behavior information and the user ID through the network and output the hair and scalp management behavior information and the user ID onto the hair diagnoser 100b.

Here, the hair and scalp management behavior information is normal, dry, intelligence, sensitivity, dandruff scalp, hair loss, each recommended shampoo information {a shampoo for a normal state, a shampoo for a dry state, a shampoo for an oily state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the normal state, the dry state, the oily state, the sensitive state, dandruff scalp, and hair loss state.

Thereafter, the behavior change analysis module 132 may control the transceiving unit 110 to receive decision making information of the user from the hair diagnoser 100b through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then store the decision making information in user unit information jointly by using a user ID as metadata.

That is, the behavior change analysis module 132 may collect big data for clear period setting for a us period of hair and scalp management behavior information by a method of collecting the decision making information of the mobile diagnoser 300 in the change step during the predetermined period in order to verify an effect by using a hair and scalp massage program providing the change step of the hair and scalp management behavior as the hair and scalp management behavior information depending on the initial hair/scalp analysis information.

The decision making information may be collected as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and collect an intention declaration representing whether to use the parameter information through an input unit of the mobile diagnoser 300.

The relevance module 133 may analyze relevance between the hair and scalp management behavior information which is the hair and scalp management behavior of the user, and each parameter information of the decision making information for the hair and scalp management behavior information which is a behavior change process characteristic.

More specifically, the relevance analysis module 133 may analyze whether to maintain each normal state, whether the dry state is changed to the normal state, whether the oily state is changed to the normal state, whether the sensitive state is changed to the normal state, whether the dandruff scalp is changed to the normal state, and whether the hair loss state is changed to the normal state corresponding to the hair and scalp management behavior information, and the initial hair/scalp analysis information depending on each parameter information of the decision making information.

To this end, the relevance analysis module 133 extracts, from the big data server 600, change parameter information (including change parameter information for a shape and a color) individually set according to the hair and the scalp when changing each hair/scalp state to the normal state for each predetermined change step (a natural number of 2 or more) to extract a predetermined change step through a comparison with a high-magnification photographing image which is image information photographed toward the scalp inside the hair received from the hair diagnoser 100b through the network between conduction depending on each change parameter information and each parameter information of one decision making information and conduction of parameter information of another decision making information.

Here, the relevance analysis module 133 stores a pattern distributively stored in the DCS DB for each state by a distribution file program on the big data server 600 and includes and stores a plurality of pattern own information for each step of each state, each state pattern inclined at a predetermined angle and a state pattern reversely inclined to extract predetermined change step information matched in a state category of the DCS DB matched by comparing the high-magnification photographing image with the plurality of pattern own information for each step of each state or each state pattern inclined at a predetermined angle and a state pattern reversely inclined.

Here, the relevance analysis module 133 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

Thereafter, in a case where one of whether to maintain the normal state and whether each state is changed to the normal state is positive and the decision making of the positive aspect (pros) and the decision making of the negative aspect (cons) opposite thereto are matched with each other in each parameter information of the decision making information for the hair and scalp management behavior information, the relevance analysis module 133 analyzes the case as positive relevance to store component information of the hair and scalp management behavior information jointly with the user unit information as the positive relevance information.

On the contrary, in a case where both whether to maintain the normal state and whether each state is changed to the normal state is negative and the decision making of the positive aspect (pros) and the decision making of the negative aspect (cons) opposite thereto are matched with each other in each parameter information of the decision making information for the hair and scalp management behavior information, the relevance analysis module 133 analyzes the case as negative relevance to store component information of the hair and scalp management behavior information jointly with the user unit information as the negative relevance information.

The model analysis module 134 analyzes relevance between the hair and scalp management characteristic of the user and a transtheoretical model configuration factor.

That is, the model analysis module 134 collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected by the relevance analysis module 133 for each user for each predetermined cycle to analyze a step indicating an intention for a behavior change of the transtheoretical model.

The transtheoretical model used in the model analysis module 134 as an integrated theoretical model for describing behaviors of individuals developed by Prochaska and DiClemente in 1983 is a mode for describing a spontaneous change of an individual behavior.

The transtheoretical model is an integrated theory for describing a principle and a process of the behavior change related to how the individuals make a new attempt for a health behavior and how to maintain the new attempt, and is to describe human behavior changes with the change step and the change process as a core. The transtheoretical model is a model that systematically integrates and configures main concepts presented in 300 or more psychotherapy theories, and does not regard the health behavior change as divided categories of success and failure, but should be appreciated as a dynamic process which is gradually occurring. That is, the individuals will gradually change the behaviors through a series of change steps. While the existing theories are just dichotomous analysis of ‘perform practice’ and ‘not perform practice’, the transtheoretical model varies depending on a research purpose for distinguishing practice performers into various steps, but is generally distinguished into 5 steps (Prochaska & Diclemente, 1983).

That is, the change step presented in the transtheoretical model as steps showing a behavior for changing a problem behavior and an intention for the behavior change is constituted by 5 steps of before plan, plan, preparation, behavior, and maintenance, and a factor which influences transition of each step to a next step is investigated, and constituted by the change step, the change process, decisional balance, and self efficacy, and the change process means strategy and technology overted and covered for modifying the problem behavior, the decisional balance is to compare and evaluate gain and loss generated to the individuals when any behavior is changed, and the self efficacy refers to confidence when any behavior change is practiced (Prochaska, J. O. Velicer, W. F., DiClemente, C. C., & Fava, J., L. 1988).

A largest advantage of the transtheoretical model is to find a feature of each step which is gradually changed rather than inducing a large change at once by analyzing an object for each step, distinguish detailed features for each step, and find a difference between pre and post steps and provide elements required for objects to progress to a next step.

That is, the model analysis module 134 controls the transceiving unit 110 to generate degree information for a change from a current hair and scalp state of the user to the normal state and generates the generated degree information as change process information, additionally receive decision making information and self efficacy information through the input unit of the hair diagnoser 100b for the user and generate step information for one of pre-plan, plan, preparation, behavior, and maintenance as a step showing an intention regarding the behavior change of the practice performer and transmit the generated step information to the mobile diagnoser 300 through the network to control to output the degree information and the step information to the output unit of the mobile diagnoser 300.

In the decisional balance, two elements of the decision making, i.e., pros and cons may be configured in the transtheoretical model, the decisional balance may mean comparing and evaluating the pros and the cons generated to the user when the user changes any behavior, and may be used as a dependent variable or an intermediary variable as a measure to confirm that the behavior change occurs and the change step is progressed.

It may be assumed that since the decision making is determined according to a degree of relative importance of the individual, a regular practice behavior is attempted or is not continued until a recognition level (pros) for the positive aspect which the regular practice gives exceeds a recognition level (cons) for the negative aspect in relation to the regular practice behavior.

That is, approval from the user for the decisional balance is a process such as recovering the confidence due to the scalp and the hair which become healthy, and on the contrary, non-approval from the user may be to avoid a changed behavior due to economic cost, and approval from the other person is to further approve the user through a complimentary remark due to a healthy and abundant scalp and hair state when the other person views the user, and on the contrary, the non-approval from the other person may be to non-approve the user a negative remark due to the healthy and abundant scalp and hair state when the other person views the user, and may be provided as a result value calculated through a multiplication for a quantitative numerical value for each of approval and non-approval items according to an input through the input unit of the hair diagnoser 100b for the approval and the non-approval for each component information of the hair and scalp management behavior information and a predetermined weight value.

The self efficacy means an individual belief that a required behavior may be successfully performed in a confront situation as a concept introduced from a self efficacy theory, and is influenced by an individual conviction degree that the component may be performed for each component information of the hair and scalp management behavior information, and it is estimated that as the self efficacy increases as the change step increases.

As a result, in the self efficacy, a multiplication of the weight value which is in proportion to a percentage of the change process information generated when the change step positively increases and a predetermined positive quantitative numerical value corresponding to each approval for the hair and the scalp of the user, and a result value of aggregating multiplied values may be generated, and a result value through multiplication for a predetermined negative quantitative numerical value corresponding to overall denial for the hair and the scalp of the user according to the weight which is in proportion to the percentage of the change process information generated when the change step negatively increases may be generated.

As a result, the model analysis module 134 controls the transceiving unit 110 to aggregate quantitative numerical values of the change process information, the decisional balance information, and the self efficacy information, and then generate step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and transmit the step information to the mobile diagnoser 300 to control the step information to the output unit of the mobile diagnoser 300.

FIG. 4 is a diagram illustrating a scalp and hair management system 1 according to yet another exemplary embodiment of the present disclosure. Referring to FIG. 4, the scalp and hair management system 1 has a structure in which a mobile terminal group 100g constituted by one or more mobile terminal 100 is connected to the hair diagnoser 100b formed for each beauty salon through short-range wireless communication, and each mobile terminal 100 is connected to a scalp and hair management server 400 and an AI server 500 through the network. Here, the mobile terminal 100 may be a form in which the hair and scalp management terminal 100a and the hair diagnoser 100b of FIG. 1 are integrated, and may correspond to a mobile device having a computing function possessed by individuals.

The network is a communication network which is a high-speed backbone network of a large communication network capable of a large-capacity, long-distance voice and data service, and may be a next-generation wired and wireless network for providing a high-speed multimedia service. When the network is a mobile communication network, the network may be a synchronous mobile communication network or an asynchronous mobile communication network. As an exemplary embodiment of the asynchronous mobile communication network, a wideband code division multiple access (WCDMA) type communication network may be used. In this case, although not illustrated in the figure, the network may include a radio network controller (RNC). Meanwhile, the WCDMA network is described as an example, but the network may be a 3G LTE network, a 4G network, a next-generation communication network such as other 5G, and an IP network based on other IPs. The network serves to mutually deliver signals and data between each mobile terminal 100, the scalp and hair management server 400, the AI server 500, and other systems.

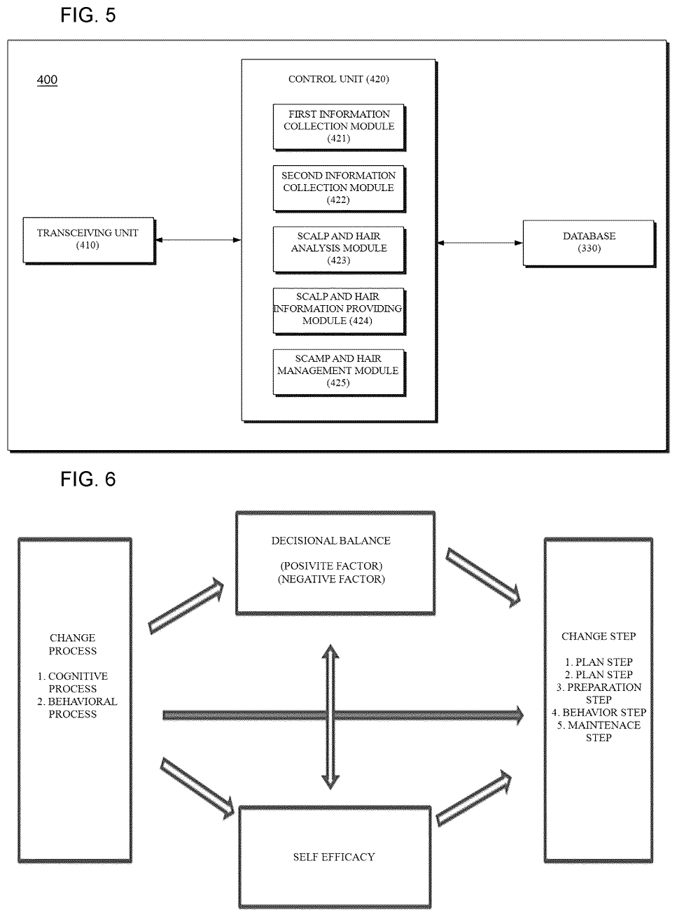

FIG. 5 is a block diagram illustrating a component of a hair and scalp management server 400 in the scalp and hair management system 1 according to the exemplary embodiment of the present disclosure.

FIG. 6 is a diagram illustrating a concept of mobile based remote scalp and hair management performed by the scalp and hair management server 400 in the scalp and hair management system 1 according to the exemplary embodiment of the present disclosure.

First, referring to FIG. 5, the scalp and hair management server 400 may include a transceiving unit 410, a control unit 420, and a database 430, and the control unit 420 may include a first information collection module 421, a second information collection module 422, a scalp and hair analysis module 423, a scalp and hair information providing module 424, and a scalp and hair management module 425.

The first information collection module 421 may control the transceiving unit 410 to receive a diagnoser ID of at least one hair diagnoser 100b of which data session is connected to the mobile terminal 100 by short-range wireless communication jointly with a first terminal identification number (IMEI) of the mobile terminal 100 according to an access from one mobile terminal 100 through the network, and then store the diagnoser ID of at least one hair diagnoser 100b in a database 430 by using the first terminal identification number (IMEI) of the mobile terminal 100 as metadata, and specify the first terminal identification number (IMEI) stored as the metadata as a manager number.

The second information collection module 422 may control the transceiving unit 410 to receive at least one diagnoser ID allocated to another mobile terminal 100 having a second terminal identification number jointly with the second terminal identification number of at least one other mobile terminal 100 for forming the same mobile terminal group 100g from the mobile terminal 100 specified as the manager number through the network, and then specify the first terminal identification number as a group name, and store both each second terminal identification number and the allocated diagnoser ID as lower category information of a specified group name.

Here, one mobile terminal 100 corresponding to the first terminal identification number may be operated by a manager who professionally manages the scalp and the hair, such as the beauty salon, a hair shop, etc., and the other mobile terminal 100 corresponding to the second terminal identification number may be operated by a user who receives management for the scalp and the hair by the manager.

In addition, the diagnoser ID which one mobile terminal 100 corresponding to the first terminal identification number registers in the database 430 may be IDs of all hair diagnosers 100b provided at a place operated by the manager, and the diagnoser ID which the other mobile terminal 100 corresponding to the second terminal identification number registers may be an ID of the mobile diagnoser 100b specified by the manager or the user among all hair diagnosers 100b provided at the place operated by the manager.

When an ID, a password, and personal information of the user are input through each mobile terminal 100, the second information collection module 422 may store the diagnoser ID of the hair diagnoser 100b specified to the user as a lower category of each terminal identification information on the database 430 as “user unit information” at the time of receiving the ID, the password, and the personal information of the user through the network.

The scalp and hair analysis module 423 may control the transceiving unit 410 to perform log-in through the user ID and the password through the network by each mobile terminal 100, and then control the transceiving unit 410 to transmit the diagnoser ID specified in the user unit information corresponding to the user ID to the mobile terminal 100 which is logged in through the network.

Thereafter, the scalp and hair analysis module 423 may control the transceiving unit 410 to receive the high-magnification photographing image of the user provided from the hair diagnoser 100b as hair/scalp state information from the logged-in mobile terminal 100 in the state of connecting the data session with the hair diagnoser 100b corresponding to the specified diagnoser ID through the short-range wireless communication, and store the high-magnification photographing image as the lower category of the user ID in the user unit information.

Here, when there is a plurality of diagnoser IDs specified to one user, the scalp and hair analysis module 423 may provide diagnoser ID information which is not used through the query for whether to use the mobile diagnoser 100b corresponding to each diagnoser ID to the mobile terminal 100 operated by the manager corresponding to the first terminal identification number to each logged-in mobile terminal 200 through the network, and herein, the mobile terminal 100 operated by the manager may automatically acquire driving state information through a management app by a short-range communication method for each hair diagnoser 100b, and then provide the acquired driving state information to the scalp and hair analysis module 423.

Thereafter, the scalp and hair analysis module 423 may generate initial hair/scalp analysis information by comparing the high-magnification photographing image with the pattern information of the normal state, the dry state, the oily state, the sensitive state, the dandruff scalp, and the hair loss state stored in the big data server 600, and then store, in the database 430, the initial hair/scalp analysis information jointly with the hair/scalp state information with the user ID as the metadata in the user unit information, and control the transceiving unit 410 to transmit the initial hair/scalp analysis information to each logged-in mobile terminal 100 to store and output the transmitted initial hair/scalp analysis information onto each logged-in mobile terminal 100.

More specifically, the scalp and hair analysis module 423 may receive, from the hair diagnoser 100b, the high-magnification photographing image which is image information acquired by shooting the scalp in the hair of the user shot by a high-magnification camera formed in the logged-in mobile terminal 100 as the hair/scalp state information, and then control the transceiving unit 410 to deliver the hair/scalp state information to the AI server 500 through the network 500 to control the transceiving unit 410 to be returned with the generated initial hair/scalp analysis information through the analysis through collection data distributively stored in the DCS DB by the distribution file program based on the big data on the AI server 500 is generated.

Here, the AI server 500 stores a pattern distributively stored in the DCS DB for each state by a distribution file program on the big data server 600 and includes and stores a plurality of pattern own information for one state, each state pattern inclined at a predetermined angle and a state pattern reversely inclined to extract a state category of the DCS DB matched by comparing the high-magnification photographing image with the plurality of pattern own information for each step of each state or each state pattern inclined at a predetermined angle and a state pattern reversely inclined as the initial hair/scalp analysis information.

The AI server 500 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

The scalp and hair analysis module 423 may extract, from the database 430, hair and scalp management behavior information depending on the analyzed initial hair/scalp analysis information, and then control the transceiving unit 410 to transmit the hair and scalp management behavior information and the user ID to the logged-in mobile terminal 100 through the network and output the hair and scalp management behavior information and the user ID onto the logged-in mobile terminal 100.

Here, the hair and scalp management behavior information is normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the normal state, the dry state, the oily state, the sensitive state, dandruff scalp, and hair loss state.

Thereafter, the scalp and hair analysis module 423 may control the transceiving unit 410 to receive decision making information of the user from each mobile terminal 100 corresponding to the second terminal identification number through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then store the decision making information in user unit information jointly by using a user ID as metadata.

That is, the scalp and hair analysis module 423 may collect big data for clear period setting for a us period of hair and scalp management behavior information by a method of collecting the decision making information from each mobile terminal 100 corresponding to the second terminal identification number in the change step during the predetermined period in order to verify an effect by using a hair and scalp massage program providing the change step of the hair and scalp management behavior as the hair and scalp management behavior information depending on the initial hair/scalp analysis information.

The decision making information may be collected as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and collect an intention declaration representing whether to use the parameter information through a touch screen which is the input unit of each mobile terminal 100 corresponding to the second terminal identification number.

The scalp and hair analysis module 423 may analyze relevance between the hair and scalp management behavior information which is the hair and scalp management behavior of the user, and each parameter information of the decision making information for the hair and scalp management behavior information which is a behavior change process characteristic.

More specifically, the scalp and hair analysis module 423 may analyze whether to maintain each normal state, whether the dry state is changed to the normal state, whether the oily state is changed to the normal state, whether the sensitive state is changed to the normal state, whether the dandruff scalp is changed to the normal state, and whether the hair loss state is changed to the normal state corresponding to the hair and scalp management behavior information, and the initial hair/scalp analysis information depending on each parameter information of the decision making information.

To this end, the AI server 500 extracts, from the big data DB, change parameter information (including change parameter information for a shape and a color) individually set according to the hair and the scalp when changing each hair/scalp state to the normal state for each predetermined change step (a natural number of 2 or more) to extract a predetermined change step through a comparison with a high-magnification photographing image which is image information photographed toward the scalp inside the hair received from the hair diagnoser 100b through the mobile terminal 100 connected to the network between conduction depending on each change parameter information and each parameter information of one decision making information and conduction of parameter information of another decision making information.

Here, the AI server 500 stores a pattern distributively stored in the DCS DB for each state by a distribution file program on the big data DB and includes and stores a plurality of pattern own information for each step of each state, each state pattern inclined at a predetermined angle and a state pattern reversely inclined to extract predetermined change step information matched in a state category of the DCS DB matched by comparing the high-magnification photographing image with the plurality of pattern own information for each step of each state or each state pattern inclined at a predetermined angle and a state pattern reversely inclined and provide the change step information to the scalp and hair analysis module 423.

Here, the AI server 500 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

Thereafter, in a case where the predetermined change step information is one of whether to maintain the normal state and whether each state is changed to the normal state is positive from a time before the hair and scalp management behavior and is matched with at least one of the decision making of the positive aspect (pros) and the decision making of the negative aspect (cons) opposite thereto in each parameter information of the decision making information for the hair and scalp management behavior information, the scalp and hair analysis module 423 analyzes the case as “the positive relevance information” to store positive component information (when the decision making of the positive aspect is each parameter information of the decision making information which influences maintaining the normal state and changing to the normal state) of the hair and scalp management behavior information and negative component information (when the decision making of the negative aspect is each parameter information of the decision making information which influences maintaining the normal state and changing to the normal state).

On the contrary, in a case where the predetermined change step information is not one of whether to maintain the normal state and whether each state is changed to the normal state is positive and is matched with at least one of the decision making of the positive aspect (pros) and the decision making of the negative aspect (cons) opposite thereto in each parameter information of the decision making information for the hair and scalp management behavior information, the scalp and hair analysis module 423 analyzes the case as “the negative relevance information” to store positive component information (when the decision making of the negative aspect is each parameter information of the decision making information which influences not maintaining the normal state and not changing to the normal state) of the hair and scalp management behavior information and negative component information (when the decision making of the positive aspect is each parameter information of the decision making information which inversely influences not maintaining the normal state and not changing to the normal state).

The scalp and hair analysis module 423 analyzes relevance between the hair and scalp management characteristic of the user and a transtheoretical model configuration factor.

That is, the scalp and hair analysis module 423 collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected by the database 430 for each user for each predetermined cycle to analyze a step indicating an intention for a behavior change of the transtheoretical model.

The transtheoretical model used in the scalp and hair analysis module 423 as an integrated theoretical model for describing behaviors of individuals developed by Prochaska and DiClemente in 1983 is a mode for describing a spontaneous change of an individual behavior.

The transtheoretical model is an integrated theory for describing a principle and a process of the behavior change related to how the individuals make a new attempt for a health behavior and how to maintain the new attempt, and is to describe human behavior changes with the change step and the change process as a core. The transtheoretical model is a model that systematically integrates and configures main concepts presented in 300 or more psychotherapy theories, and does not regard the health behavior change as divided categories of success and failure, but should be appreciated as a dynamic process which is gradually occurring. That is, the individuals will gradually change the behaviors through a series of change steps. While the existing theories are just dichotomous analysis of ‘perform practice’ and ‘not perform practice’, the transtheoretical model varies depending on a research purpose for distinguishing practice performers into various steps, but is generally distinguished into 5 steps (Prochaska & Diclemente, 1983).

That is, the change step presented in the transtheoretical model as steps showing a behavior for changing a problem behavior and an intention for the behavior change is constituted by 5 steps of before plan, plan, preparation, behavior, and maintenance, and a factor which influences transition of each step to a next step is investigated, and constituted by the change step, the change process, decision making balance, and self efficacy, and the change process means strategy and technology overted and covered for modifying the problem behavior, the decision making balance is to compare and evaluate gain and loss generated to the individuals when any behavior is changed, and the self efficacy refers to confidence when any behavior change is practiced (Prochaska, J. O. Velicer, W. F., DiClemente, C. C., & Fava, J., L. 1988).

A largest advantage of the transtheoretical model is to find a feature of each step which is gradually changed rather than inducing a large change at once by analyzing an object for each step, distinguish detailed features for each step, and find a difference between pre and post steps and provide elements required for objects to progress to a next step.

That is, the scalp and hair analysis module 423 controls the transceiving unit 410 to generate degree information for a change from a current hair and scalp state of the user to the normal state and generates the generated degree information as change process information, additionally receive decision making information and self efficacy information through the input unit of the mobile terminal 100 for the user and generate step information for one of pre-plan, plan, preparation, behavior, and maintenance as a step showing an intention regarding the behavior change of the practice performer and transmit the generated step information to the mobile terminal 100 through the network to control to output the degree information and the step information to the output unit of the mobile diagnoser 300.

In the decisional balance, two elements of the decision making, i.e., pros and cons may be configured in the transtheoretical model, the decisional balance may mean comparing and evaluating the pros and the cons generated to the user when the user changes any behavior, and may be used as a dependent variable or an intermediary variable as a measure to confirm that the behavior change occurs and the change step is progressed.

It may be assumed that since the decision making is determined according to a degree of relative importance of the individual, a regular practice behavior is attempted or is not continued until a recognition level (pros) for the positive aspect which the regular practice gives exceeds a recognition level (cons) for the negative aspect in relation to the regular practice behavior.

That is, approval from the user for the decisional balance is a process such as recovering the confidence due to the scalp and the hair which become healthy, and on the contrary, non-approval from the user may be to avoid a changed behavior due to economic cost, and approval from the other person is to further approve the user through a complimentary remark due to a healthy and abundant scalp and hair state when the other person views the user, and on the contrary, the non-approval from the other person may be to non-approve the user a negative remark due to the healthy and abundant scalp and hair state when the other person views the user, and may be provided as a result value calculated through a multiplication for a quantitative numerical value for each of approval and non-approval items according to an input through the input unit of the mobile terminal 100 for the approval and the non-approval for each component information of the hair and scalp management behavior information and a predetermined weight value.

The self efficacy means an individual belief that a required behavior may be successfully performed in a confront situation as a concept introduced from a self efficacy theory, and is influenced by an individual conviction degree that the component may be performed for each component information of the hair and scalp management behavior information, and it may be estimated that as the self efficacy increases as the change step increases.

As a result, in the self efficacy, a multiplication of the weight value which is in proportion to a percentage of the change process information generated when the change step positively increases and a predetermined positive quantitative numerical value corresponding to each approval for the hair and the scalp of the user, and a result value of aggregating multiplied values may be generated, and a result value through multiplication for a predetermined negative quantitative numerical value corresponding to overall denial for the hair and the scalp of the user according to the weight which is in proportion to the percentage of the change process information generated when the change step negatively increases may be generated.

As a result, the scalp and hair analysis module 423 may aggregate quantitative numerical values of the change process information, the decisional balance information, and the self efficacy information, and then generate step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and store the step information in the database 430.

The scalp and hair information providing module 424 controls the transceiving unit 410 to transmit information on five steps of pre-plan, plan, preparation, behavior, and maintenance as the change steps presented in the transtheoretical model stored in the database 430 to the mobile terminal 100 through the network to control the information to be output to the touch screen which is the output unit of the mobile terminal 100.

The scalp and hair information providing module 424 may extract, as first information, positive component information (when the decision making of the positive aspect is each parameter information of the decision making information which influences maintaining the normal state and changing to the normal state) of the hair and scalp management behavior information and negative component information (when the decision making of the negative aspect is each parameter information of the decision making information which influences maintaining the normal state and changing to the normal state) analyzed as the “positive relevance” stored in the database 430 by the scalp and hair analysis module 423.

The scalp and hair information providing module 424 may extract, as second information, positive component information (when the decision making of the negative aspect is each parameter information of the decision making information which influences not maintaining the normal state and not changing to the normal state) of the hair and scalp management behavior information and negative component information (when the decision making of the positive aspect is each parameter information of the decision making information which inversely influences not maintaining the normal state and not changing to the normal state) analyzed as the “negative relevance” stored in the database 430 by the scalp and hair analysis module 423.

Thereafter, the scalp and hair information providing module 424 may generate graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal 100 is represented as a timeline between neighboring steps, and provide the graph information to the mobile terminal 100 operated by the user who manages the scalp and the hair corresponding to the second terminal identification number and the mobile terminal 100 operated by the manager corresponding to the first terminal identification number.

The scalp and hair management module 425 controls the transceiving unit 410 to extract component information which is most frequently generated among the positive component information of the positive relevance and the negative component information of the negative relevance during moving to a higher step in information on first steps of pre-plan, plan, preparation, behavior, and maintenance as the change steps presented in the theoretical model and transmit the component information to the mobile terminal 100 through the network as recommended behavior information most suitable for the user to provide the most suitable recommended behavior information for continuation to a next step and manage the user.

Moreover, the scalp and hair management module 425 controls the transceiving unit 410 to extract component information which is most frequently generated among the negative component information of the positive relevance and the positive component information of the negative relevance during moving to a lower step in information on first steps of pre-plan, plan, preparation, behavior, and maintenance as the change steps presented in the theoretical model and transmit the component information to the mobile terminal 100 through the network as non-recommended behavior information most suitable for the user to provide behavior information to be most avoided not to return to a previous step and manage the user.

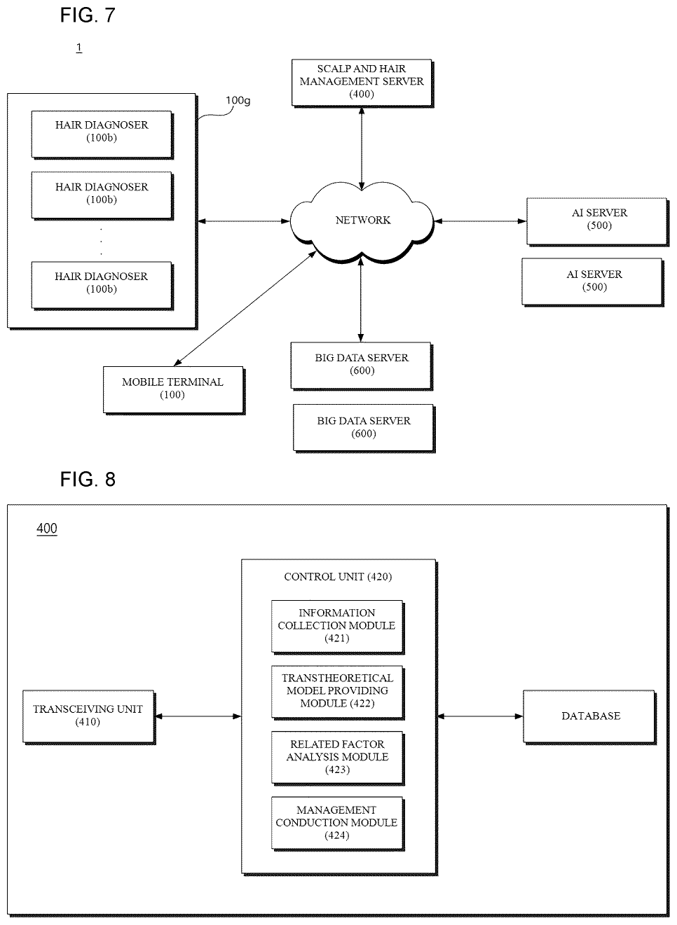

FIG. 7 is a diagram illustrating a scalp and hair management system 1 according to yet another exemplary embodiment of the present disclosure. Referring to FIG. 7, the scalp and hair management system 1 may include the mobile terminal 100, a hair diagnoser group 100bg constituted by a plurality of hair diagnosers 100b, the network, the scalp and hair management server 400, the AI server 500, and the big data server 600.

The network is a communication network which is a high-speed backbone network of a large communication network capable of a large-capacity, long-distance voice and data service, and may be a next-generation wired and wireless network for providing a high-speed multimedia service. When the network is a mobile communication network, the network may be a synchronous mobile communication network or an asynchronous mobile communication network. As an exemplary embodiment of the asynchronous mobile communication network, a wideband code division multiple access (WCDMA) type communication network may be used. In this case, although not illustrated in the figure, the network may include a radio network controller (RNC). Meanwhile, the WCDMA network is described as an example, but the network may be a 3G LTE network, a 4G network, a next-generation communication network such as other 5G, and an IP network based on other IPs. The network serves to mutually transfer signals and data among the mobile terminal 100, the hair diagnoser group 100bg constituted by the plurality of hair diagnosers 100b, the scalp and hair management server 400, the AI server 500, the big data server 600, and other systems.

FIG. 8 is a block diagram illustrating a component of a hair and scalp management server 400 in the scalp and hair management system 1 according to another exemplary embodiment of the present disclosure. First, referring to FIG. 8, the scalp and hair management server 400 may include a transceiving unit 410, a control unit 420, and a database 430, and the control unit 420 may include an information collection module 421, a transtheoretical model providing module 422, a related factor analysis module 423, and management conduction module 424.

When an ID, a password, and personal information of the user for use for each hair diagnoser 100b constituting the hair diagnoser group 100bg are received from the mobile terminal 100 through the network, the information collection module 422 may store the ID, the password, and the personal information of the user on the database 430 as the user unit information.

Thereafter, the information collection module 421 may control the transceiving unit 410 to perform log-in through the user ID and the password through the network by the mobile terminal 100, control the transceiving unit 410 to receive the high-magnification photographing image of the user from the hair diagnoser 100b corresponding to the diagnoser ID as the hair and scalp state information (hereinafter, referred to as hair/scalp state information) when the diagnoser ID of one hair diagnoser 100b constituting the hair diagnoser group 100bg is received from the mobile terminal 100 through the network, and store the high-magnification photographing image by using the user ID as the metadata in the user unit information.

Thereafter, the transtheoretical model providing module 422 may receive the initial hair/scalp analysis information from the AI server 500 through an initial hair/scalp analysis request for the AI server 500 through the network for the high-magnification photographing image.

Here, the AI server 500 may generate initial hair/scalp analysis information by comparing the high-magnification photographing image with the pattern information of the normal state, the dry state, the oily state, the sensitive state, the dandruff scalp, and the hair loss state stored in the big data server 600 through the network, and then provide the initial hair/scalp analysis information to the transtheoretical model providing module 422 to store the initial hair/scalp analysis information jointly with the hair/scalp state information with the user ID as the metadata in the user unit information on the database 430 by the transtheoretical model providing module 422, and transmit the initial hair/scalp analysis information to the mobile terminal 100 by the transtheoretical model providing module 422 and output the transmitted initial hair/scalp analysis information to the output unit of the mobile terminal 100.

More specifically, the AI server 500 may receive the high-magnification photographing image which is image information acquired by shooting the scalp in the hair of the user shot by a high-magnification camera formed in the hair diagnoser 100b as the hair/scalp state information, and then generate the initial hair/scalp analysis information through analysis of collected data distributed and stored in a DCS DB and a machine learning algorithm by the hair/scalp state information and a distributed file program on the data server 500.

Here, the big data server 600 stores patterns distributed and stored in the DCS DB for each state by a distribution file program and the big data server 600, stores, in at least one DCS DB, a plurality of pattern own information for each state or information on each state pattern which is inclined at a predetermined angle, and information on each state pattern which is inversely inclined, and as a result, the AI server 500 may extract state category of the DCS DB matching by comparing the information of the DCS DB and the high-magnification photographing image as the initial hair/scalp analysis information.

The AI server 500 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

The transtheoretical model providing module 422 may extract, from the database 430, hair and scalp management behavior information depending on the analyzed initial hair/scalp analysis information, and then provide the hair and scalp management behavior information and the user ID to the mobile terminal 100 through the network and output the hair and scalp management behavior information and the user ID to the output unit of the mobile terminal 100.

Here, the hair and scalp management behavior information may include each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the analyzed initial hair/scalp analysis information corresponding to the normal state, the dry state, the oily state, the sensitive state, dandruff scalp, and hair loss state.

Thereafter, the transtheoretical model providing module 422 may control the transceiving unit 410 to receive decision making information of the user from the hair diagnoser 100b through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then store the decision making information in user unit information jointly by using a user ID as metadata.

That is, the transtheoretical model providing module 422 may collect big data for clear period setting for a us period of hair and scalp management behavior information by a method of collecting the decision making information from the mobile terminal 100 in the change step during the predetermined period in order to verify an effect by using a hair and scalp massage program providing the change step of the hair and scalp management behavior as the hair and scalp management behavior information depending on the initial hair/scalp analysis information.

The decision making information may be collected as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and collect an intention declaration representing whether to use the parameter information through an input unit of the mobile diagnoser 300.

The transtheoretical model providing module 422 may analyze relevance between the hair and scalp management behavior information which is the hair and scalp management behavior of the user, and each parameter information of the decision making information for the hair and scalp management behavior information which is a behavior change process characteristic.

More specifically, the transtheoretical model providing module 422 may analyze whether to maintain each normal state, whether the dry state is changed to the normal state, whether the oily state is changed to the normal state, whether the sensitive state is changed to the normal state, whether the dandruff scalp is changed to the normal state, and whether the hair loss state is changed to the normal state corresponding to the hair and scalp management behavior information, and the initial hair/scalp analysis information depending on each parameter information of the decision making information.

To this end, the AI server 500 extracts, from the big data server 600, change parameter information (including change parameter information for a shape and a color) individually set according to the hair and the scalp when changing each hair/scalp state to the normal state for each predetermined change step (a natural number of 2 or more) to extract a predetermined change step through a comparison with a high-magnification photographing image which is image information photographed toward the scalp inside the hair received from the hair diagnoser 100b through the transtheoretical model providing module 422 between conduction depending on each change parameter information and each parameter information of one decision making information and conduction of parameter information of another decision making information.

Here, a pattern distributively stored in the DCS DB is stored for each state by a distribution file program on the big data server 600 and includes and stores a plurality of pattern own information for each step of each state, the AI server 500 extracts predetermined change step information matched in a state category of the DCS DB matched by comparing the high-magnification photographing image with the plurality of pattern own information for each step of each state or each state pattern inclined at a predetermined angle and a state pattern reversely inclined.

Further, the AI server 500 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

Thereafter, in a case where one of whether to maintain the normal state and whether each state is changed to the normal state is positive received from the AI server 500 and the decision making of the positive aspect (pros) and the decision making of the negative aspect (cons) opposite thereto are matched with each other in each parameter information of the decision making information for the hair and scalp management behavior information, the transtheoretical model providing module 422 analyzes the case as positive relevance to store component information of the hair and scalp management behavior information jointly with the user unit information as the positive relevance information.

On the contrary, in a case where both whether to maintain the normal state and whether each state is changed to the normal state is negative and the decision making of the positive aspect (pros) and the decision making of the negative aspect (cons) opposite thereto are matched with each other in each parameter information of the decision making information for the hair and scalp management behavior information, the transtheoretical model providing module 422 analyzes the case as negative relevance to store component information of the hair and scalp management behavior information jointly with the user unit information as the negative relevance information.

The transtheoretical model providing module 422 analyzes relevance between the hair and scalp management characteristic of the user and a transtheoretical model configuration factor.

That is, the transtheoretical model providing module 422 collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected on the database 430 for each user for each predetermined cycle to analyze a step indicating an intention for a behavior change of the transtheoretical model.

The transtheoretical model used in the transtheoretical model providing module 422 as an integrated theoretical model for describing behaviors of individuals developed by Prochaska and DiClemente in 1983 is a mode for describing a spontaneous change of an individual behavior.

The transtheoretical model is an integrated theory for describing a principle and a process of the behavior change related to how the individuals make a new attempt for a health behavior and how to maintain the new attempt, and is to describe human behavior changes with the change step and the change process as a core. The transtheoretical model is a model that systematically integrates and configures main concepts presented in 300 or more psychotherapy theories, and does not regard the health behavior change as divided categories of success and failure, but should be appreciated as a dynamic process which is gradually occurring. That is, the individuals will gradually change the behaviors through a series of change steps. While the existing theories are just dichotomous analysis of ‘perform practice’ and ‘not perform practice’, the transtheoretical model varies depending on a research purpose for distinguishing practice performers into various steps, but is generally distinguished into 5 steps (Prochaska & Diclemente, 1983).

That is, the change step presented in the transtheoretical model as steps showing a behavior for changing a problem behavior and an intention for the behavior change is constituted by 5 steps of before plan, plan, preparation, behavior, and maintenance, and a factor which influences transition of each step to a next step is investigated, and constituted by the change step, the change process, decision making balance, and self efficacy, and the change process means strategy and technology overted and covered for modifying the problem behavior, the decision making balance is to compare and evaluate gain and loss generated to the individuals when any behavior is changed, and the self efficacy refers to confidence when any behavior change is practiced (Prochaska, J. O. Velicer, W. F., DiClemente, C. C., & Fava, J., L. 1988).

A largest advantage of the transtheoretical model is to find a feature of each step which is gradually changed rather than inducing a large change at once by analyzing an object for each step, distinguish detailed features for each step, and find a difference between pre and post steps and provide elements required for objects to progress to a next step.

That is, the transtheoretical model providing module 422 controls the transceiving unit 410 to generate degree information for a change from a current hair and scalp state of the user to the normal state and generates the generated degree information as change process information, additionally receive decision making information and self efficacy information through the input unit of the mobile terminal 100 for the user and generate step information for one of pre-plan, plan, preparation, behavior, and maintenance as a step showing an intention regarding the behavior change of the practice performer and transmit the generated step information to the mobile terminal 100 through the network to control to output the degree information and the step information to the output unit of the mobile terminal 100.

In the decisional balance, two elements of the decision making, i.e., pros and cons may be configured in the transtheoretical model, the decisional balance may mean comparing and evaluating the pros and the cons generated to the user when the user changes any behavior, and may be used as a dependent variable or an intermediary variable as a measure to confirm that the behavior change occurs and the change step is progressed.

It may be assumed that since the decision making is determined according to a degree of relative importance of the individual, a regular practice behavior is attempted or is not continued until a recognition level (pros) for the positive aspect which the regular practice gives exceeds a recognition level (cons) for the negative aspect in relation to the regular practice behavior.

That is, approval from the user for the decisional balance is a process such as recovering the confidence due to the scalp and the hair which become healthy, and on the contrary, non-approval from the user may be to avoid a changed behavior due to economic cost, and approval from the other person is to further approve the user through a complimentary remark due to a healthy and abundant scalp and hair state when the other person views the user, and on the contrary, the non-approval from the other person may be to non-approve the user a negative remark due to the healthy and abundant scalp and hair state when the other person views the user, and may be provided as a result value calculated through a multiplication for a quantitative numerical value for each of approval and non-approval items according to an input through the input unit of the mobile terminal 100 for the approval and the non-approval for each component information of the hair and scalp management behavior information and a predetermined weight value.

The self efficacy means an individual belief that a required behavior may be successfully performed in a confront situation as a concept introduced from a self efficacy theory, and is influenced by an individual conviction degree that the component may be performed for each component information of the hair and scalp management behavior information, and it is estimated that as the self efficacy increases as the change step increases.

As a result, in the self efficacy, a multiplication of the weight value which is in proportion to a percentage of the change process information generated when the change step positively increases and a predetermined positive quantitative numerical value corresponding to each approval for the hair and the scalp of the user, and a result value of aggregating multiplied values may be generated, and a result value through multiplication for a predetermined negative quantitative numerical value corresponding to overall denial for the hair and the scalp of the user according to the weight which is in proportion to the percentage of the change process information generated when the change step negatively increases may be generated.

As a result, the transtheoretical model providing module 422 controls the transceiving unit 410 to aggregate quantitative numerical values of the change process information, the decisional balance information, and the self efficacy information, and then generate step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and transmit the step information to the mobile terminal 100 to control the step information to the output unit of the mobile terminal 100.

When the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the decisional balance information in order to determine the first to fifth range steps to provide the decisional balance information, approval item information extracted through extraction is provided to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the transtheoretical model providing module 422 to know that a positive mind of the user progresses the movement to the higher step.

On the contrary, when the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the decisional balance information in order to determine the first to fifth range steps to provide the decisional balance information, approval item information extracted through extraction is provided to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the transtheoretical model providing module 422 to know that a negative mind of the user progresses the movement to the lower step.

When the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the self efficacy information to provide the self efficacy information, approval item information extracted through extraction is provided to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the transtheoretical model providing module 422 to know that the positive mind of the user progresses the movement to the higher step.

On the contrary, when the related factor analysis module 423 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the self efficacy information to provide the self efficacy information, non-approval item information extracted through extraction is provided to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the transtheoretical model providing module 422 to know that the negative mind of the user progresses the movement to the lower step.

The management conduction module 424 may extract, as first information, positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state) analyzed as the “positive relevance” stored in the database 430 for each user ID.

Further, the management conduction module 424 may extract, as second information, positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state) analyzed as the “negative relevance” stored in the database 430.

Thereafter, the management conduction module 424 may generate graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal 100 is represented as a timeline between neighboring steps, and provide approval item information (first information) extracted through extraction with respect to response to the positive aspect in the decisional balance information and the approval item for forming the decisional balance information during moving to the higher step in information on five steps of pre-plan, plan, preparation, behavior, and maintenance as the change steps presented in the theoretical model presented by the related factor analysis module 423 and approval item information (second information) extracted through extraction with respect to response to the negative aspect in the decisional balance information and the approval items for forming the decisional balance information during moving to the lower step in information on five steps of pre-plan, plan, preparation, behavior, and maintenance as the change steps presented in the theoretical model to the mobile terminal 100 through the network for each user ID in real time.

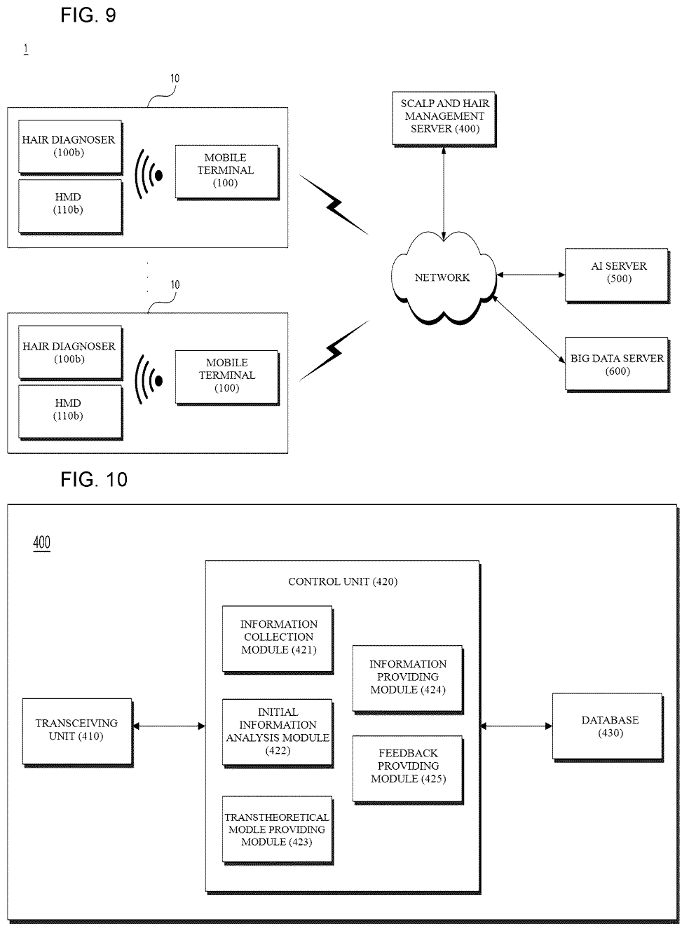

FIG. 9 is a diagram illustrating a scalp and hair management system 1 according to yet another exemplary embodiment of the present disclosure. Referring to FIG. 9, the scalp and hair management system 1 may include a user personal terminal group constituted by a plurality of user personal terminals 10, the network, the scalp and hair management server 400, the AI server 500, and the big data server 600, and each user personal terminal 10 may include the mobile terminal 100, the hair diagnoser 100b, and an HMD 110b.

The network is a communication network which is a high-speed backbone network of a large communication network capable of a large-capacity, long-distance voice and data service, and may be a next-generation wired and wireless network for providing a high-speed multimedia service. When the network is a mobile communication network, the network may be a synchronous mobile communication network or an asynchronous mobile communication network. As an exemplary embodiment of the asynchronous mobile communication network, a wideband code division multiple access (WCDMA) type communication network may be used. In this case, although not illustrated in the figure, the network may include a radio network controller (RNC). Meanwhile, the WCDMA network is described as an example, but the network may be a 3G LIE network, a 4G network, a next-generation communication network such as other 5G, and an IP network based on other IPs. The network serves to mutually deliver signals and data between each user personal terminal 10, the scalp and hair management server 400, the AI server 500, the big data server 600, and other systems.

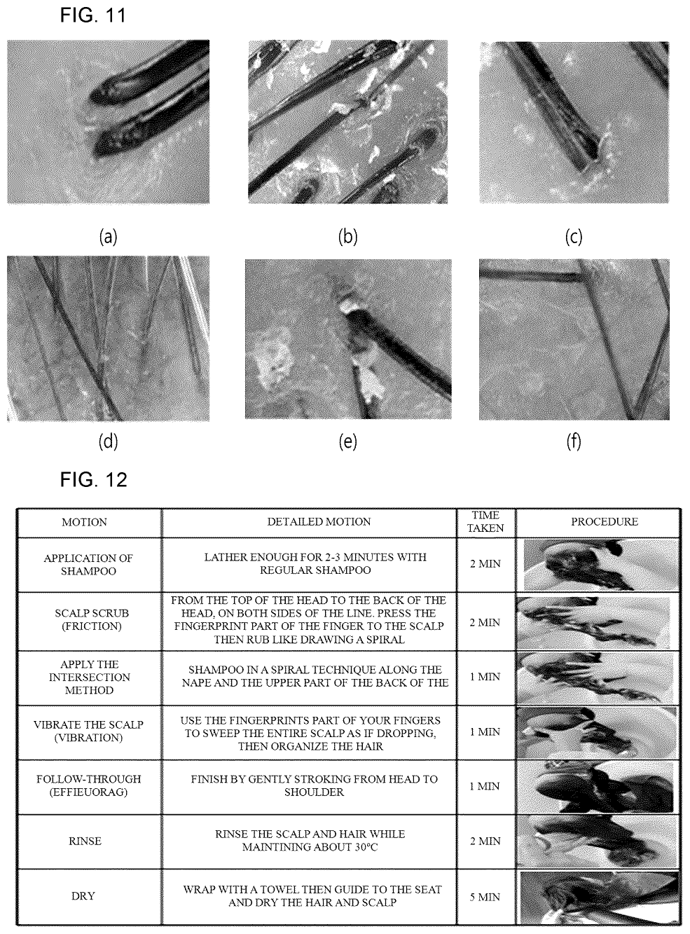

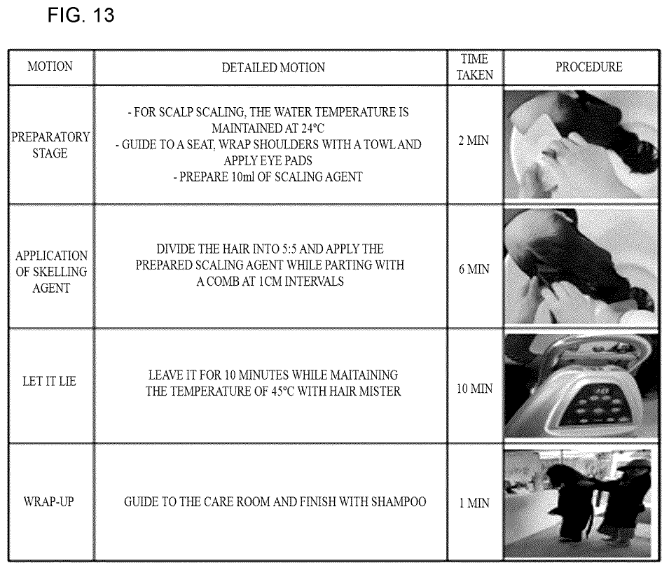

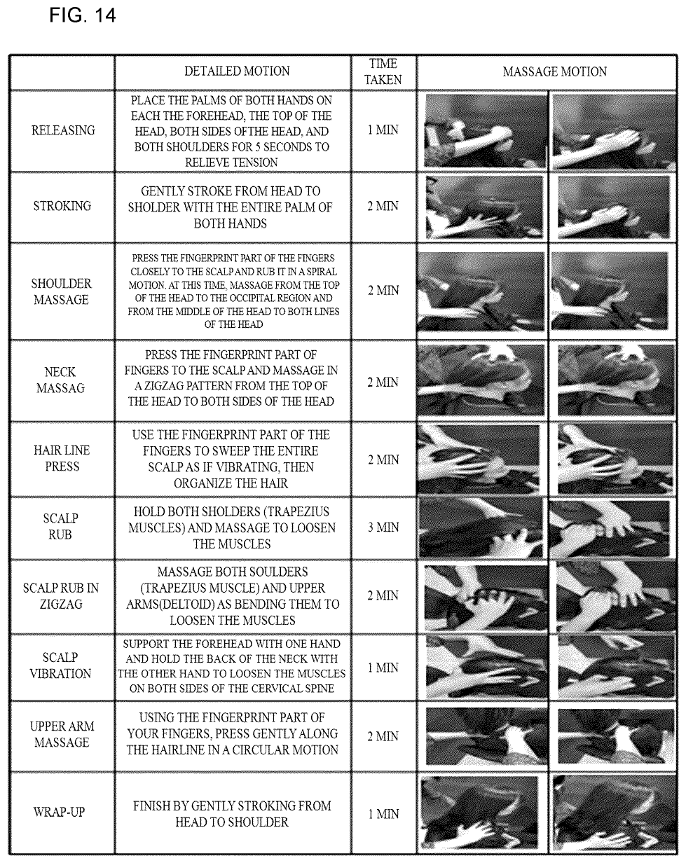

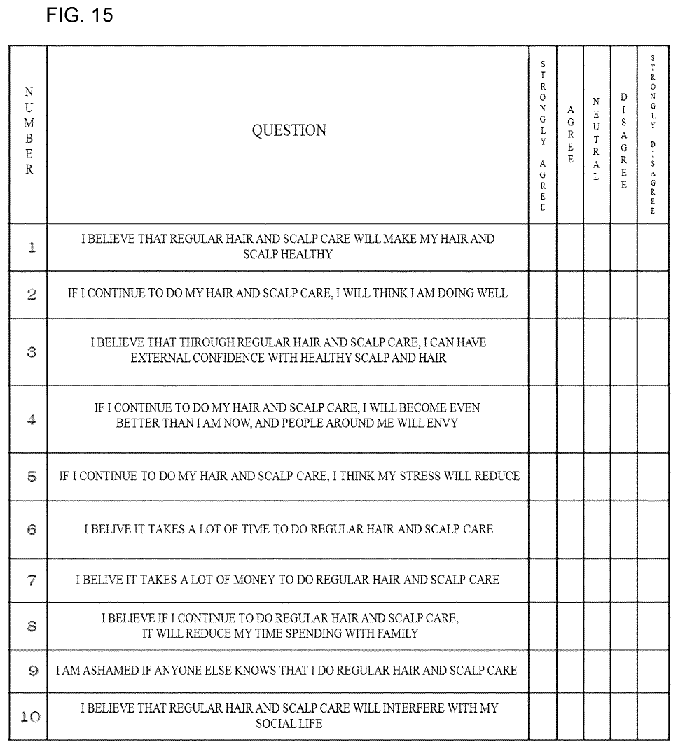

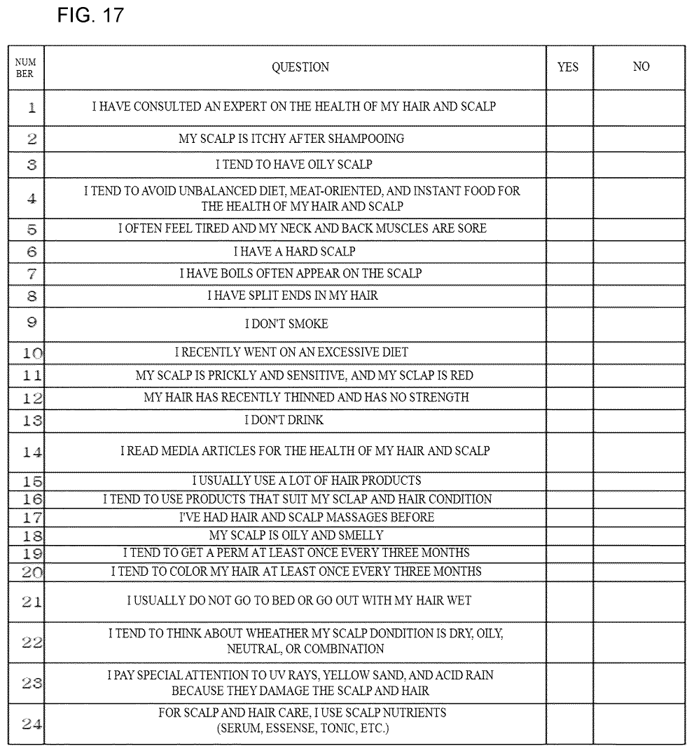

FIG. 10 is a block diagram illustrating a component of a hair and scalp management server 400 in the scalp and hair management system 1 according to still yet another exemplary embodiment of the present disclosure. FIG. 11 is a diagram for describing pattern information of a normal state (FIG. 1A), a dry state (FIG. 11B), an oily state (FIG. 11C), a sensitive state (FIG. 11), a dandruff scalp (FIG. 11E), and a hair loss state (FIG. 11F) by an AI server 500 in the scalp and hair management system 1 according to still yet another exemplary embodiment of the present disclosure. FIGS. 12 to 14 are diagrams for describing each stage information of one recommended shampoo information (FIG. 12) in hair and scalp management action information provided by the scalp and hair management server 400 (FIG. 12), each stage information included in one scalp scaling information in the hair and scalp management action information (FIG. 12), each stage information included in one scalp scaling information in the hair and scalp management action information (FIG. 13), and one scalp massage information in the hair and scalp management action information (FIG. 14) in the scalp and hair management system 1 according to still yet another exemplary embodiment of the present disclosure. FIGS. 15 to 17 are diagrams for describing information collection of collecting information by the scalp and hair management server 400 in the scalp and hair management system 1 according to still another exemplary embodiment of the present disclosure.

First, referring to FIG. 10, the scalp and hair management server 400 may include the transceiving unit 410, the control unit 420, and the database 430, and the control unit 420 may include the information collection module 421, the initial information analysis module 422, the transtheoretical model providing module 423, the information providing module 424, and a feedback providing module 425.

The information collection module 421 may control the transceiving unit 410 to receive a diagnoser ID of at least one hair diagnoser 100b and an HMD ID of the HMD 100a of which data session is connected to the mobile terminal 100 and the HMD 100a by short-range wireless communication jointly with the terminal identification number (IMEI) of the mobile terminal 100 according to an access from one mobile terminal 100 through the network, and then store the diagnoser ID and the HMD ID in the database 430.

In addition, each of the diagnoser ID and the HMD ID which one mobile terminal 100 corresponding to the terminal identification number registers may perform the short-range wireless communication with the mobile terminal 100 with a personal smart device operated by the user.

When an ID and a password of the user are input through each mobile terminal 100, the information collection module 422 may store each terminal identification number as the metadata by using the user ID, the password, the diagnoser ID, and the HMD ID on the database 430 as the “user unit information” at the time of receiving the ID and the password of the user through the network.

Further, the information collection module 421 may control the transceiving unit 410 to perform the log-in through the user ID and the password through the network by the mobile terminal 100, control the transceiving unit 410 to receive the high-magnification photographing image of the user from the scalp and hair diagnoser 100 connected to the mobile terminal 100 by the short-range wireless communication from the mobile terminal 100 as the hair and scalp state information (hereinafter, referred to as hair/scalp state information), control the transceiving unit 410 to receive brainwave information from a brainwave measurement device (not illustrated) connected to the mobile terminal 100 by the short-range wireless communication, and store the high-magnification photographing image (hair/scalp state information) and initial reference brainwave information by using the user ID as the metadata in the user unit information.

The initial information analysis module 422 may receive the initial hair/scalp analysis information from the AI server 500 through an initial hair/scalp analysis request for the AI server 500 through the network for the high-magnification photographing image.

Here, the AI server 500 may generate initial hair/scalp analysis information by comparing the high-magnification shooting image with the pattern information of the normal state (FIG. 11A), the dry state (FIG. 11B), the oily state (FIG. 11C), the sensitive state (FIG. 11D), the dandruff scalp (FIG. 11E), and the hair loss state (FIG. 11F) stored in the big data server 600 through the network, and then provide the initial hair/scalp analysis information to the initial information analysis module 422 to store the initial hair/scalp analysis information jointly with the high-magnification photographing image (the hair/scalp state information) and the brainwave information with the user ID as the metadata in the user unit information on the database 430 by the initial information analysis module 422, and transmit the initial hair/scalp analysis information to the mobile terminal 100 by the initial information analysis module 422 and output the transmitted initial hair/scalp analysis information to the output unit of the mobile terminal 100.

More specifically, the AI server 500 may receive the high-magnification shooting image which is image information acquired by shooting the scalp in the hair of the user shot by a high-magnification camera formed in the scalp and hair diagnoser 100 as the hair/scalp state information, and then generate the initial hair/scalp analysis information through analysis of collected data distributed and stored in a DCS DB and a machine learning algorithm by the hair/scalp state information and a distributed file program on the data server 500.

Here, the big data server 600 stores patterns distributed and stored in the DCS DB for each state by a distribution file program and the big data server 600, stores, in at least one DCS DB, a plurality of pattern own information for each state or information on each state pattern which is inclined at a predetermined angle, and information on each state pattern which is inversely inclined, and as a result, the AI server 500 may extract state category of the DCS DB matching by comparing the information of the DCS DB and the high-magnification shooting image as the initial hair/scalp analysis information.

The AI server 500 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

Here, the initial information analysis module 422 may receive, from the mobile terminal 100, brainwave change information from a brainwave measurer connected to the mobile terminal 100 by the short-range wireless communication at the time of outputting the initial hair/scalp analysis information to the output unit of the mobile terminal 100 according to the transmission of the initial hair/scalp analysis information to the mobile terminal 100, and store each terminal identification number as the metadata on the database 430 with first change brainwave information as the “user unit information”.

The initial information analysis module 422 may extract, from the database 430, hair and scalp management behavior information depending on the analyzed initial hair/scalp analysis information, and then provide the hair and scalp management behavior information and the user ID to the mobile terminal 100 through the network and output the hair and scalp management behavior information and the user ID to the output unit of the mobile terminal 100.

Here, the hair and scalp management behavior information may include each recommended shampoo information {a shampoo for a normal state, a shampoo for a dry state, a shampoo for an oily state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the analyzed initial hair/scalp analysis information corresponding to the normal state (FIG. 11A), the dry state (FIG. 11B), the oily state (FIG. 11C), the sensitive state (FIG. 11D), the dandruff scalp (FIG. 11E).

Meanwhile, each step information of one recommended shampoo information in the hair and scalp management behavior information may be provided by a scheme in FIG. 12, each step of one scalp scaling information in the hair and scalp management behavior information may also be provided by a scheme in FIG. 13, and one scalp massage information in the hair and scalp management behavior information may be provided by a scheme in FIG. 14.

The transtheoretical model providing module 423 may control the transceiving unit 410 to receive decision making information of the user from the scalp and hair diagnoser 100 through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then store the decision making information in user unit information jointly by using the user ID as the metadata.

That is, the transtheoretical model providing module 423 may collect big data for clear period setting for a us period of hair and scalp management behavior information by a method of collecting the decision making information from the mobile terminal 100 in the change step during the predetermined period in order to verify an effect by using a hair and scalp massage program providing the change step of the hair and scalp management behavior as the hair and scalp management behavior information depending on the initial hair/scalp analysis information.

Here, the transtheoretical model providing module 423 controls the transceiving unit 410 to receive the high-magnification photographing image (hair/scalp state information) collected by the hair diagnoser 100b from the mobile terminal 100 through the network for each predetermined cycle (e.g., 3 days, 1 week, etc.) within a predetermined period in order to collect the decision making information, and then provides the high-magnification photographing image (hair/scalp state information) jointly with received intermediate analysis result information for each cycle to generate the decision making information as provided reaction information (response and brainwave information to O and X through the input unit) according to each query.

Here, the transtheoretical model providing module 423 may collect, from the mobile terminal connected to a brainwave measurement device, brainwave change information according to a first query for decision making of the positive aspect (pros) according to the use of each recommended shampoo information/decision making of the negative aspect (cons) opposite thereto, brainwave change information according to a second query for decision making of the positive aspect (pros) according to the use of each scalp scaling information/decision making of the negative aspect (cons) opposite thereto, and brainwave change information according to a third query for decision making of the positive aspect (pros) according to the use of scalp massage information/decision making of the negative aspect (cons) opposite thereto, as brainwave information.

Meanwhile, the decision making information may be collected as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information.

Here, the first to third parameter information may be collected by a scheme of providing the brainwave change information measured by the brainwave measurement device from the transtheoretical model providing module 423 to the mobile terminal 100 as reference information, and receiving whether to use the component constituting each hair and scalp management behavior information from the mobile terminal 100 for an intention expression (O and X) of the user through the input unit of the mobile terminal 100.

The brainwave change information provided to the mobile terminal 100 by the transtheoretical model providing module 423 may be analyzed by decision making of a negative aspect (cons) opposite thereto when a predetermined frequency or more increases in each initial reference brainwave information and a positive aspect (pros) when the predetermined frequency or more does not increase for a user who stares at the intermediate analysis result information for each cycle while the initial reference brainwave information is primarily classified into a delta wave, a theta wave, an alpha wave, a beta wave, and an gamma wave.

Meanwhile, in this process, in order to provide the intermediate analysis result information to the mobile terminal 100, the transtheoretical model providing module 423 may analyze whether to maintain each normal state (FIG. 11A), whether the dry state (FIG. 11B) is changed to the normal state, whether the oily state (FIG. 11C) is changed to the normal state, whether the sensitive state (FIG. 11D) is changed to the normal state, whether the dandruff scalp (FIG. 11E) is changed to the normal state, and whether the hair loss state (FIG. 11F) is changed to the normal state corresponding to the hair and scalp management behavior information, and the initial hair/scalp analysis information depending on each parameter information of the decision making information.

To this end, the AI server 500 extracts, from the big data server 600, change parameter information (including change parameter information for a shape and a color) individually set according to the hair and the scalp when changing each hair/scalp state to the normal state for each predetermined change step (a natural number of 2 or more) to extract a predetermined change step through a comparison with a high-magnification photographing image which is image information photographed toward the scalp inside the hair received from the scalp and hair diagnoser 100 through the initial information analysis module 422 between conduction depending on each change parameter information and each parameter information of one decision making information and conduction of parameter information of another decision making information.

Here, a pattern distributively stored in the DCS DB is stored for each state by a distribution file program on the big data server 600 and includes and stores a plurality of pattern own information for each step of each state, the AI server 500 extracts predetermined change step information matched in a state category of the DCS DB matched by comparing the high-magnification photographing image with the plurality of pattern own information for each step of each state or each state pattern inclined at a predetermined angle and a state pattern reversely inclined.

Further, the AI server 500 may extract characteristics of a pixel from features of the pixel for hair and scalp regions, i.e., brightness of the pixel, a color of the pixel, a line formed by the pixel, and a form of the pixel which is changed according to the elapse of the time, in addition to the type of form, and determine a pixel corresponding to a state pattern including a characteristic similar to the characteristic of each pixel jointly as a similar range.

Meanwhile, in a case where one of whether to maintain the normal state (FIG. 11A) and whether each state is changed to the normal state is positive received from the AI server 500 and the decision making of the positive aspect (pros) and the decision making of the negative aspect (cons) opposite thereto are matched with each other in each parameter information of the decision making information for the hair and scalp management behavior information, the transtheoretical model providing module 423 analyzes the case as positive relevance to store component information of the hair and scalp management behavior information jointly with the user unit information as the positive relevance information.

On the contrary, in a case where both whether to maintain the normal state (FIG. 11A) and whether each state is changed to the normal state is negative and the decision making of the positive aspect (pros) and the decision making of the negative aspect (cons) opposite thereto are matched with each other in each parameter information of the decision making information for the hair and scalp management behavior information, the transtheoretical model providing module 423 analyzes the case as negative relevance to store component information of the hair and scalp management behavior information jointly with the user unit information as the negative relevance information.

The transtheoretical model providing module 423 analyzes relevance between the hair and scalp management characteristic of the user and a transtheoretical model configuration factor.

That is, the transtheoretical model providing module 423 collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected on the database 430 for each user for each predetermined cycle to analyze a step indicating an intention for a behavior change of the transtheoretical model.

The transtheoretical model used in the transtheoretical model providing module 423 as an integrated theoretical model for describing behaviors of individuals developed by Prochaska and DiClemente in 1983 is a mode for describing a spontaneous change of an individual behavior.

The transtheoretical model is an integrated theory for describing a principle and a process of the behavior change related to how the individuals make a new attempt for a health behavior and how to maintain the new attempt, and is to describe human behavior changes with the change step and the change process as a core. The transtheoretical model is a model that systematically integrates and configures main concepts presented in 300 or more psychotherapy theories, and does not regard the health behavior change as divided categories of success and failure, but should be appreciated as a dynamic process which is gradually occurring. That is, the individuals will gradually change the behaviors through a series of change steps. While the existing theories are just dichotomous analysis of ‘perform practice’ and ‘not perform practice’, the transtheoretical model varies depending on a research purpose for distinguishing practice performers into various steps, but is generally distinguished into 5 steps (Prochaska & Diclemente, 1983).

That is, the change step presented in the transtheoretical model as steps showing a behavior for changing a problem behavior and an intention for the behavior change is constituted by 5 steps of before plan, plan, preparation, behavior, and maintenance, and a factor which influences transition of each step to a next step is investigated, and constituted by the change step, the change process, decision making balance, and self efficacy, and the change process means strategy and technology overted and covered for modifying the problem behavior, the decision making balance is to compare and evaluate gain and loss generated to the individuals when any behavior is changed, and the self efficacy refers to confidence when any behavior change is practiced (Prochaska, J. O. Velicer, W. F., DiClemente, C. C., & Fava, J., L. 1988).

A largest advantage of the transtheoretical model is to find a feature of each step which is gradually changed rather than inducing a large change at once by analyzing an object for each step, distinguish detailed features for each step, and find a difference between pre and post steps and provide elements required for objects to progress to a next step.

That is, the transtheoretical model providing module 423 controls the transceiving unit 410 to generate degree information for a change from a current hair and scalp state of the user to the normal state and generates the generated degree information as change process information, additionally receive decision making information and self efficacy information through the input unit of the mobile terminal 100 for the user and generate step information for one of pre-plan, plan, preparation, behavior, and maintenance as a step showing an intention regarding the behavior change of the practice performer and transmit the generated step information to the mobile terminal 100 through the network to control to output the degree information and the step information to the output unit of the mobile terminal 100.

In the decisional balance, two elements of the decision making, i.e., pros and cons may be configured in the transtheoretical model, the decisional balance may mean comparing and evaluating the pros and the cons generated to the user when the user changes any behavior, and may be used as a dependent variable or an intermediary variable as a measure to confirm that the behavior change occurs and the change step is progressed.

It may be assumed that since the decision making is determined according to a degree of relative importance of the individual, a regular practice behavior is attempted or is not continued until a recognition level (pros) for the positive aspect which the regular practice gives exceeds a recognition level (cons) for the negative aspect in relation to the regular practice behavior.

That is, approval from the user for the decisional balance is a process such as recovering the confidence due to the scalp and the hair which become healthy, and on the contrary, non-approval from the user may be to avoid a changed behavior due to economic cost, and approval from the other person is to further approve the user through a complimentary remark due to a healthy and abundant scalp and hair state when the other person views the user, and on the contrary, the non-approval from the other person may be to non-approve the user a negative remark due to the healthy and abundant scalp and hair state when the other person views the user, and may be provided as a result value calculated through a multiplication for a quantitative numerical value for each of approval and non-approval items according to an input through the input unit of the mobile terminal 100 for the approval and the non-approval for each component information of the hair and scalp management behavior information and a predetermined weight value.

The self efficacy means an individual belief that a required behavior may be successfully performed in a confront situation as a concept introduced from a self efficacy theory, and is influenced by an individual conviction degree that the component may be performed for each component information of the hair and scalp management behavior information, and it is estimated that as the self efficacy increases as the change step increases.

As a result, in the self efficacy, a multiplication of the weight value which is in proportion to a percentage of the change process information generated when the change step positively increases and a predetermined positive quantitative numerical value corresponding to each approval for the hair and the scalp of the user, and a result value of aggregating multiplied values may be generated, and a result value through multiplication for a predetermined negative quantitative numerical value corresponding to overall denial for the hair and the scalp of the user according to the weight which is in proportion to the percentage of the change process information generated when the change step negatively increases may be generated.

As a result, the transtheoretical model providing module 423 controls the transceiving unit 410 to aggregate quantitative numerical values of the change process information, the decisional balance information, and the self efficacy information, and then generate step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and transmit the step information to the mobile terminal 100 to control the step information to the output unit of the mobile terminal 100.

When the information providing module 424 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the decisional balance in order to determine the first to fifth range steps to provide the decisional balance information, approval item information extracted through extraction is provided to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the initial information analysis module 422 to know that a positive mind of the user progresses the movement to the higher step.

On the contrary, when the information providing module 424 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all as illustrated in FIG. 15 like not YES or NO for each of the approval and non-approval items for the decisional balance in order to determine the first to fifth range steps to provide the decisional balance information, approval item information extracted through extraction is provided to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the initial information analysis module 422 to know that a negative mind of the user progresses the movement to the lower step.

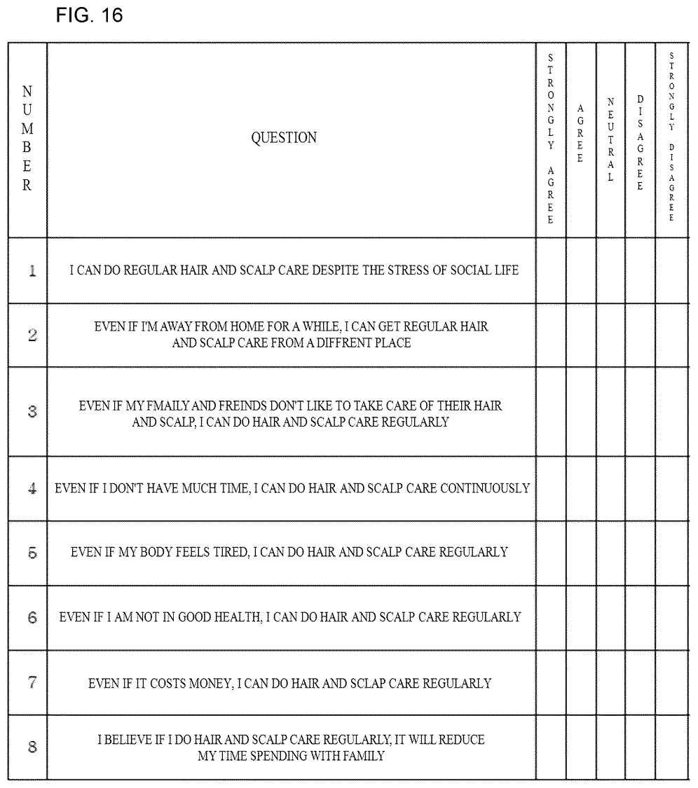

When the information providing module 424 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) as illustrated in FIG. 16 like very so, sometimes so, so, not almost so, and so not at all like not YES or NO for each of the approval and non-approval items for the self efficacy information to provide the self efficacy information, approval item information extracted through extraction is provided to the mobile terminal 100 through the network jointly with the user ID with respect to response to the positive aspect in the approval item during moving to the higher step in information on five steps of pre-plan, plan, preparation, behavior, and maintenance as the change steps presented in the theoretical model by the initial information analysis module 422 to know that the positive mind of the user progresses the movement to the higher step.

On the contrary, when the information providing module 424 aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) as illustrated in FIG. 16 like very so, sometimes so, so, not almost so, and so not at all like not YES or NO for each of the approval and non-approval items for the self efficacy information to provide the self efficacy information, non-approval item information extracted through extraction is provided to the mobile terminal 100 through the network jointly with the user ID with respect to response to the positive aspect in the non-approval item during moving to the lower step in information on five steps of pre-plan, plan, preparation, behavior, and maintenance as the change steps presented in the theoretical model by the initial information analysis module 422 to know that the negative mind of the user progresses the movement to the lower step.

The information providing module 424 may extract, as first information, positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state) analyzed as the “positive relevance” stored in the database 430 for each user ID.

Further, the information providing module 424 may extract, as second information, positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state) analyzed as the “negative relevance” stored in the database 430.

Thereafter, the information providing module 424 may generate graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal 100 is represented as a timeline between neighboring steps, and provide approval item information (first information) extracted through extraction with respect to response to the positive aspect in the decisional balance information and the approval item for forming the decisional balance information during moving to the higher step in information on five steps of pre-plan, plan, preparation, behavior, and maintenance as the change steps presented in the theoretical model presented by the information providing module 424 and approval item information (second information) extracted through extraction with respect to response to the negative aspect in the decisional balance information and the approval items for forming the decisional balance information during moving to the lower step in information on five steps of pre-plan, plan, preparation, behavior, and maintenance as the change steps presented in the theoretical model to the mobile terminal 100 through the network for each user ID in real time.

The feedback providing module 425 may generate a VR image according to the timeline for a 2D image for the high-magnification photographing image (hair/scalp state information) for generating the intermediate analysis result information for each cycle from an initially collected high-magnification photographing image (hair/scalp state information), and provide the generated VR image to the mobile terminal 100 through the network.

In order to generate the VR image according to the cycle of each timeline, the feedback providing module 425 may determine a plurality of focal positions and focuses for the high-magnification photographing image which is the 2D image, and compute a focal distance between respective focal positions for a plurality of focus 2D image data and compute a depth value which is in inverse proportion to the focal distance computed between the respective focal positions when a plurality of multiple focus 2D image data corresponding to the plurality of focus positions and focuses determined.

Thereafter, the feedback providing module 425 performs decoding of extracting and informationizing image information (a color, chroma, and brightness) corresponding to each pixel for a range of image data corresponding to each focus number of the high-magnification photographing image (hair/scalp state information) to generate decoded data and generate a polygon set to which a depth value computed for each pixel is reflected for a polygon which is a basic unit for expressing the decoded data as a 3D shape and then performs pixel mapping of attaching the decoded data onto the polygon set to generate each specified focus-specific virtual reality (VR) image.

Thereafter, the feedback providing module 425 provides the set of the VR image according to the cycle of each timeline to the mobile terminal 100 through the network to provide the scalp and hair state according to the hair and scalp management behavior information which is a behavior change process as the time elapses to the user as a realistic VR image through the HMD 100a connected to the mobile terminal 100 through the short-range wireless communication.

The present disclosure relates to a scalp and hair management system, and more particularly, to a scalp and hair management system which: has increased precision by collecting user information, particularly decision-making information and electroencephalographic information so as to provide aspects of change in scalp and hair managing habits in order to apply a transtheoretical model relating to scalp and hair managing habits for users; provides, by means of realistic image information, changed state information in accordance with systemic management of scalp and hair; verifies effects of using multiple scalp and hair management programs in stages of changes of the scalp and hair management from an initial state; finds and provides a management program suitable for a user; analyzes and provides positive factors and negative factors of the management program; provides state information of the user in stages; and thus boosts motivation for management.

What is claimed is:

1. A scalp and hair management system comprising: a hair and scalp management terminal; a network; and a hair diagnoser group constituted by a plurality of hair diagnosers, wherein the hair and scalp management terminal includes
a member joining module which generates initial hair/scalp analysis information by comparing the high-magnification photographing image with the pattern information of the normal state, the dry state, the oily state, the sensitive state, the dandruff scalp, and the hair loss state stored in the big data server, and then stores, in the storage unit, the initial hair/scalp analysis information jointly with the hair/scalp state information with the user ID as the metadata in the user unit information, and provides the mobile diagnoser with the initial hair/scalp analysis information and outputs the provided initial hair/scalp analysis information onto the hair diagnoser,
stores patterns distributed and stored in the DCS DB for each state by a distribution file program and the big data server, stores, in at least one DCS DB, a plurality of pattern own information for each state or information on each state pattern which is inclined at a predetermined angle, and information on each state pattern which is inversely inclined, and extracts state category of the DCS DB matching by comparing the information of the DCS DB and the high-magnification shooting image as the initial hair/scalp analysis information, and
extracts characteristics of a pixel from features of the pixel for hair and scalp regions, i.e., brightness of the pixel, a color of the pixel, a line formed by the pixel, and a form of the pixel which is changed according to the elapse of the time, in addition to the form, and determines a pixel corresponding to a state pattern including a characteristic similar to the characteristic of each pixel as a similar range,
a behavior change analysis module which extracts hair and scalp management behavior information according to the initial hair/scalp analysis information analyzed by the member joining module from the storage unit, and then provide the hair and scalp management behavior information and the user ID to the hair diagnoser through the network, and outputs the hair and scalp management behavior information onto the hair diagnoser and provides the hair and scalp management behavior information including normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the normal state, the dry state, the oily state, the sensitive state, dandruff scalp, and hair loss state,
controls the transceiving unit to receive decision making information of the user from the hair diagnoser through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then stores the decision making information in user unit information jointly by using a user ID as metadata,
collects big data for clear period setting for a us period of hair and scalp management behavior information by a method of collecting the decision making information of the mobile diagnoser in the change step during the predetermined period in order to verify an effect by using a hair and scalp massage program providing the change step of the hair and scalp management behavior as the hair and scalp management behavior information depending on the initial hair/scalp analysis information, and
collects the decision making information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and receives information collected through the input unit of the mobile diagnoser when collecting the information, and
a relevance analysis module which analyzes relevance between the hair and scalp management behavior information which is the hair and scalp management behavior of the user, and each parameter information of the decision making information for the hair and scalp management behavior information which is a behavior change process characteristic, and
extracts, from the big data server, change parameter information (including change parameter information for a shape and a color) individually set according to the hair and the scalp when changing each hair/scalp state to the normal state for each predetermined change step (a natural number of 2 or more) to extract a predetermined change step through a comparison with a high-magnification photographing image which is image information photographed toward the scalp inside the hair received from the hair diagnoser through the network between conduction depending on each change parameter information and each parameter information of one decision making information and conduction of parameter information of another decision making information.

2. The scalp and hair management system of claim 1, wherein the member subscription module stores an ID, a password, and personal information of the user when the ID, the password, and the personal information of the user are input through the input/output unit, and controls the transceiving unit to perform log-in through the user ID and password through the network by one hair diagnoser constituting the mobile diagnoser group, and then controls the transceiving unit to receive a high-magnification photographing image of the user from the hair diagnoser as hair/scalp state information, and stores the high-magnification photographing image with the user ID as metadata in the user unit information.

3. A scalp and hair management system 1 having a structure in which a mobile terminal group constituted by one or more mobile terminals is connected to the hair diagnoser formed for each beauty salon through short-range wireless communication, and each mobile terminal is connected to a scalp and hair management server, wherein the scalp and hair management server includes
a first information collection module which controls the transceiving unit to receive a diagnoser ID of at least one hair diagnoser of which data session is connected to the mobile terminal by short-range wireless communication jointly with a first terminal identification number (IMEI) of the mobile terminal according to an access from one mobile terminal through the network, and then stores the diagnoser ID of at least one hair diagnoser in a database by using the first terminal identification number (IMEI) of the mobile terminal as metadata, and specifies the first terminal identification number (IMEI) stored as the metadata as a manager number,
a second information collection module which control the transceiving unit to receive at least one diagnoser ID allocated to another mobile terminal having a second terminal identification number jointly with the second terminal identification number of at least one other mobile terminal for forming the same mobile terminal group from the mobile terminal specified as the manager number through the network, and then specify the first terminal identification number as a group name, and store both each second terminal identification number and the allocated diagnoser ID as lower category information of a specified group name, wherein one mobile terminal corresponding to the first terminal identification number may be operated by a manager who professionally manages the scalp and the hair, such as the beauty salon, a hair shop, etc., and the other mobile terminal corresponding to the second terminal identification number may be operated by a user who receives management for the scalp and the hair by the manager, and the diagnoser ID which one mobile terminal corresponding to the first terminal identification number registers in the database may be IDs of all hair diagnosers provided at a place operated by the manager, and the diagnoser ID which the other mobile terminal corresponding to the second terminal identification number registers may be an ID of the mobile diagnoser specified by the manager or the user among all hair diagnosers provided at the place operated by the manager, and
stores, when an ID, a password, and personal information of the user are input through each mobile terminal, the diagnoser ID of the hair diagnoser specified to the user as a lower category of each terminal identification information on the database as “user unit information” at the time of receiving the ID, the password, and the personal information of the user through the network, and
a scalp and hair analysis module which controls the transceiving unit to perform log-in through the user ID and the password through the network by each mobile terminal, and then controls the transceiving unit to transmit the diagnoser ID specified in the user unit information corresponding to the user ID to the mobile terminal which is logged in through the network,
controls the transceiving unit to receive the high-magnification photographing image of the user provided from the hair diagnoser as hair/scalp state information from the logged-in mobile terminal in the state of connecting the data session with the hair diagnoser corresponding to the specified diagnoser ID through the short-range wireless communication, and stores the high-magnification photographing image as the lower category of the user ID in the user unit information,
provides, when there is a plurality of diagnoser IDs specified to one user, diagnoser ID information which is not used through the query for whether to use the mobile diagnoser corresponding to each diagnoser ID to the mobile terminal operated by the manager corresponding to the first terminal identification number to each logged-in mobile terminal through the network, wherein when the mobile terminal operated by the manager automatically acquires driving state information through a management app by a short-range communication method for each hair diagnoser, receives the acquired driving state information from the mobile terminal,
generates initial hair/scalp analysis information by comparing the high-magnification shooting image with the pattern information of the normal state, the dry state, the oily state, the sensitive state, the dandruff scalp, and the hair loss state stored in the big data server, and then stores, in the database, the initial hair/scalp analysis information jointly with the hair/scalp state information with the user ID as the metadata in the user unit information, and controls the transceiving unit to transmit the initial hair/scalp analysis information to each logged-in mobile terminal to store and output the transmitted initial hair/scalp analysis information onto each logged-in mobile terminal,
receives, from the hair diagnoser, the high-magnification shooting image which is image information acquired by photographing the scalp in the hair of the user shot by a high-magnification camera formed in the logged-in mobile terminal as the hair/scalp state information, and then controls the transceiving unit to deliver the hair/scalp state information to the AI server through the network to control the transceiving unit to be returned with the generated initial hair/scalp analysis information through the analysis through collection data distributively stored in the DCS DB by the distribution file program based on the big data on the AI server is generated,
extracts, from the database, hair and scalp management behavior information depending on the analyzed initial hair/scalp analysis information, and then controls the transceiving unit to transmit the hair and scalp management behavior information and the user ID to the logged-in mobile terminal through the network and output the hair and scalp management behavior information and the user ID onto the logged-in mobile terminal,
controls the transceiving unit 410 to receive decision making information of the user from each mobile terminal 100 corresponding to the second terminal identification number through the network for each predetermined cycle within a predetermined period when conducting the behavior change analysis during a predetermined period in order to determine a behavior change process depending on a change step depending on hair and scalp management behavior information conduction depending on initial hair/scalp analysis information, and then stores the decision making information in user unit information jointly by using a user ID as metadata,
collects big data for clear period setting for a us period of hair and scalp management behavior information by a method of collecting the decision making information from each mobile terminal corresponding to the second terminal identification number in the change step during the predetermined period in order to verify an effect by using a hair and scalp massage program providing the change step of the hair and scalp management behavior as the hair and scalp management behavior information depending on the initial hair/scalp analysis information,
collects the decisional information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information, and collects an intention declaration representing whether to use the parameter information through a touch screen which is the input unit of each mobile terminal corresponding to the second terminal identification number,
analyzes relevance between the hair and scalp management behavior information which is the hair and scalp management behavior of the user, and each parameter information of the decision making information for the hair and scalp management behavior information which is a behavior change process characteristic though a request for the AI server,
in a case where the predetermined change step information is one of whether to maintain the normal state and whether each state is changed to the normal state is positive from a time before the hair and scalp management behavior and is matched with at least one of the decision making of the positive aspect (pros) and the decision making of the negative aspect (cons) opposite thereto in each parameter information of the decision making information for the hair and scalp management behavior information, analyzes the case as “the positive relevance information” to store positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state),
on the contrary, in a case where the predetermined change step information is not one of whether to maintain the normal state and whether each state is changed to the normal state is positive and is matched with at least one of the decision making of the positive aspect (pros) and the decision making of the negative aspect (cons) opposite thereto in each parameter information of the decision making information for the hair and scalp management behavior information, analyzes the case as “the negative relevance information” to store positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state),
analyzes relevance between the hair and scalp management characteristic of the user and a transtheoretical model configuration factor, and collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected by the database for each user for each predetermined cycle to analyze a step indicating an intention for a behavior change of the transtheoretical model, and controls the transceiving unit to generate degree information for a change from a current hair and scalp state of the user to the normal state and generates the generated degree information as change process information, additionally receive decision making information and self efficacy information through the input unit of the mobile terminal for the user and generate step information for one of pre-plan, plan, preparation, behavior, and maintenance as a step showing an intention regarding the behavior change of the practice performer and transmit the generated step information to the mobile terminal through the network to control to output the degree information and the step information to the output unit of the mobile diagnoser,
wherein in the decisional balance, two elements of the decision making, i.e., pros and cons may be configured in the transtheoretical model, the decisional balance may mean comparing and evaluating the pros and the cons generated to the user when the user changes any behavior, and may be used as a dependent variable or an intermediary variable as a measure to confirm that the behavior change occurs and the change step is progressed,
it is assumed that since the decision making is determined according to a degree of relative importance of the individual, a regular practice behavior is attempted or is not continued until a recognition level (pros) for the positive aspect which the regular practice gives exceeds a recognition level (cons) for the negative aspect in relation to the regular practice behavior, and provided as a result value calculated through a multiplication for a quantitative numerical value for each of approval and non-approval items according to an input through the input unit of the mobile terminal for the approval and the non-approval for each component information of the hair and scalp management behavior information and a predetermined weight value,
in the self efficacy, a multiplication of the weight value which is in proportion to a percentage of the change process information generated when the change step positively increases and a predetermined positive quantitative numerical value corresponding to each approval for the hair and the scalp of the user, and a result value of aggregating multiplied values may be generated, and a result value through multiplication for a predetermined negative quantitative numerical value corresponding to overall denial for the hair and the scalp of the user according to the weight which is in proportion to the percentage of the change process information generated when the change step negatively increases may be generated, and
aggregates quantitative numerical values of the change process information, the decisional balance information, and the self efficacy information, and then generates step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and stores the step information in the database.

4. The scalp and hair management system of claim 3, wherein the hair and scalp management behavior information includes each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each of the normal state, the dry state, the oily state, the sensitive state, dandruff scalp, and hair loss state.

5. The scalp and hair management system of claim 3, wherein the scalp and hair management server further includes
a scalp and hair management module which controls the transceiving unit to extract component information which is most frequently generated among the positive component information of the positive relevance and the negative component information of the negative relevance during moving to a higher step in information on first steps of pre-plan, plan, preparation, behavior, and maintenance as the change steps presented in the theoretical model and transmit the component information to the mobile terminal through the network as recommended behavior information most suitable for the user to provide the most suitable recommended behavior information for continuation to a next step and manage the user.

6. A scalp and hair management system including a mobile terminal, a hair diagnoser group constituted by a plurality of hair diagnosers, a network, and a scalp and hair management server, wherein the scalp and hair management server includes
a transtheoretical model providing module, and
a related factor analysis module which provides approval item information extracted through extraction to the mobile terminal through the network jointly with the user ID with respect to response to the positive aspect in the approval item during moving to the higher step in information on five steps of pre-plan, plan, preparation, behavior, and maintenance as the change steps presented in the theoretical model by the transtheoretical model providing module to know that a positive mind of the user progresses the movement to the higher step, when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all like not YES or NO for each of the approval and non-approval items for the decisional balance information to provide the decisional balance information in order to determine first to fifth range steps of five steps of pre-plan, plan, preparation, behavior, and maintenance as the change steps presented in the transtheoretical model according to user information analysis.

7. The scalp and hair management system of claim 6, wherein when the related factor analysis module aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the decisional balance in order to determine the first to fifth range steps to provide the decisional balance information, approval item information extracted through extraction is provided to the mobile terminal through the network jointly with the user ID with respect to response to the positive aspect in the non-approval item during moving to the lower step in information on five steps of pre-plan, plan, preparation, behavior, and maintenance as the change steps presented in the theoretical model by the transtheoretical model providing module to know that a negative mind of the user progresses the movement to the lower step.

8. The scalp and hair management system of claim 7, wherein when the related factor analysis module aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, approval item information extracted through extraction is provided to the mobile terminal through the network jointly with the user ID with respect to response to the positive aspect in the approval item during moving to the higher step in information on five steps of pre-plan, plan, preparation, behavior, and maintenance as the change steps presented in the theoretical model by the transtheoretical model providing module to know that a positive mind of the user causes the movement to the higher step.

9. The scalp and hair management system of claim 8, wherein when the related factor analysis module aggregates numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, non-approval item information extracted through extraction is provided to the mobile terminal through the network jointly with the user ID with respect to response to the positive aspect in the non-approval item during moving to the lower step in information on five steps of pre-plan, plan, preparation, behavior, and maintenance as the change steps presented in the theoretical model by the transtheoretical model providing module to know that a negative mind of the user causes the movement to the lower step.

10. The scalp and hair management system of claim 9, wherein the scalp and hair management server further includes a management conduction module which extracts, as first information, positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state) analyzed as the “positive relevance” stored in the database for each user ID.

11. The scalp and hair management system of claim 10, wherein the management conduction module extracts, as second information, positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state) analyzed as the “negative relevance” stored in the database.

12. The scalp and hair management system of claim 11, wherein the management conduction module generates graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal is represented as a timeline between neighboring steps.

13. The scalp and hair management system of claim 12, wherein the management conduction module provides approval item information (first information) extracted through extraction with respect to response to the positive aspect in the decisional balance information and the approval item for forming the decisional balance information during moving to the higher step in information on five steps of pre-plan, plan, preparation, behavior, and maintenance as the change steps presented in the theoretical model presented by the related factor analysis module and approval item information (second information) extracted through extraction with respect to response to the negative aspect in the decisional balance information and the approval items for forming the decisional balance information during moving to the lower step in information on five steps of pre-plan, plan, preparation, behavior, and maintenance as the change steps presented in the theoretical model to the mobile terminal through the network for each user ID in real time.

14. The scalp and hair management system of claim 13, wherein the transtheoretical model providing module receives the initial hair/scalp analysis information from the AI server through an initial hair/scalp analysis request for the AI server through the network for the high-magnification photographing image included in the hair and scalp state information (hereinafter, referred to hair/scalp state information) of the user provided from the hair diagnoser.

15. The scalp and hair management system of claim 14, wherein the transtheoretical model providing module extracts hair and scalp management behavior information according to the analyzed initial hair/scalp analysis information from the database, and then provide the hair and scalp management behavior information and the user ID to the mobile terminal through the network, and outputs the hair and scalp management behavior information onto the mobile terminal and provides the hair and scalp management behavior information including normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (including the number of times of massage and the cycle of the message according to the state, etc.) for each analyzed initial hair/scalp analysis information corresponding to the normal state, the dry state, the oily state, the sensitive state, dandruff scalp, and hair loss state.

16. The scalp and hair management system of claim 15, wherein the transtheoretical model providing module controls the transceiving unit to receive decision making information of the user from the hair diagnoser through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then stores the decision making information in user unit information jointly by using a user ID as metadata.

17. The scalp and hair management system of claim 16, wherein the transtheoretical model providing module collects the decision making information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information,
analyzes relevance between the hair and scalp management behavior information which is the hair and scalp management behavior of the user, and each parameter information of the decision making information for the hair and scalp management behavior information which is a behavior change process characteristic though a request for the AI server,
in a case where one of whether to maintain the normal state and whether each state is changed to the normal state received from the AI server is positive and the decision making of the positive aspect (pros) and the decision making of the negative aspect (cons) opposite thereto are matched with each other in each parameter information of the decision making information for the hair and scalp management behavior information, analyzes the case as positive relevance to store component information of the hair and scalp management behavior information jointly with the user unit information as the positive relevance information,
in a case where both whether to maintain the normal state and whether each state is changed to the normal state is negative and the decision making of the positive aspect (pros) and the decision making of the negative aspect (cons) opposite thereto are matched with each other in each parameter information of the decision making information for the hair and scalp management behavior information, analyzes the case as negative relevance to store component information of the hair and scalp management behavior information jointly with the user unit information as the negative relevance information, and
analyzes relevance with hair and scalp management characteristics of the user and a transtheoretical model configuration factor.

18. A scalp and hair management system 1 comprising a user personal terminal group constituted by a plurality of user personal terminals, a network, a scalp and hair management server, an AI server, and a big data server, wherein each user personal terminal 10 includes a mobile terminal, a hair diagnoser, and an HMD, and each of a diagnoser ID and an HMD ID which one mobile terminal corresponding to a terminal identification number registers in a database performs short-range wireless communication with the mobile terminal with a personal smart device operated by a user, wherein the scalp and hair management server includes
an information collection module which controls the transceiving unit to receive a diagnoser ID of at least one hair diagnoser and an HMD ID of the HMD of which data session is connected to the mobile terminal and the HMD from one mobile terminal jointly with the terminal identification number (IMEI) of the mobile terminal according to an access through the network, and then stores the diagnoser ID and the HMD ID by using the terminal identification number (IMEI) of the mobile terminal as metadata in the database, controls the transceiving unit to store each terminal identification number as the metadata by using the user ID, the password, the diagnoser ID, and the HMD ID on the database as the “user unit information” at the time of receiving the ID and the password of the user through the network and perform log-in through the user ID and the password through the network by the mobile terminal when an ID and a password of the user are input through each mobile terminal, controls the transceiving unit to receive the high-magnification photographing image of the user from the scalp and hair diagnoser connected to the mobile terminal by short-range wireless communication from the mobile terminal as hair and scalp state information (hereinafter, referred to as hair/scalp state information) and controls the transceiving unit to receive brainwave information from a brainwave measurement device connected to the mobile terminal by the short-range wireless communication, and stores the high-magnification photographing image (hair/scalp state information) and initial reference brainwave information by using the user ID as the metadata in the user unit information,
a transtheoretical model providing module which receives the initial hair/scalp analysis information from the AI server through an initial hair/scalp analysis request for the AI server through the network for the high-magnification photographing image,
receives, from the mobile terminal, brainwave change information from a brainwave measurer connected to the mobile terminal by the short-range wireless communication at the time of outputting the initial hair/scalp analysis information to the output unit of the mobile terminal according to the transmission of the initial hair/scalp analysis information to the mobile terminal, and stores each terminal identification number as the metadata on the database with first change brainwave information as the “user unit information”,
extracts hair and scalp management behavior information according to the analyzed initial hair/scalp analysis information from the database, and then provide the hair and scalp management behavior information and the user ID to the mobile terminal through the network, and outputs the hair and scalp management behavior information onto the mobile terminal and provides the hair and scalp management behavior information including normal, dry, intelligence, sensitivity, dandruff scalp, hair loss, each recommended shampoo information {a shampoo for a normal state, a shampoo for a dry state, a shampoo for an oil state, a shampoo for a sensitive state, an anti-dandruff shampoo, and a hair loss shampoo}, scalp scaling information {the number of times and a cycle of scalp scaling according to each state; option information of a first step of removal of keratin, sebum, and wastes accumulated in the scalp and the hair according to each state, a second step of ampoule input (product selection by dividing the oily and dry scalp scaling according to the state), a third step of activation (cleaning) of a capillary vessel, etc.}, scalp massage information (the number of times of massage and the cycle of the message according to the state, etc.) for each analyzed initial hair/scalp analysis information corresponding to the normal state, the dry state, the oily state, the sensitive state, dandruff scalp, and hair loss state,
controls the transceiving unit to receive decision making information of the user from the scalp and hair diagnoser 100 through the network for each predetermined cycle (e.g., 3 days, 1 week, etc.) within a predetermined period when conducting the behavior change analysis during a predetermined period (e.g., 6 months to 5 years) in order to determine a behavior change process depending on a change step depending on hair and scalp management behavior information conduction depending on initial hair/scalp analysis information, and then stores the decision making information in user unit information jointly by using a user ID as metadata,
controls the transceiving unit to receive the high-magnification photographing image (hair/scalp state information) collected by the hair diagnoser from the mobile terminal through the network for each predetermined cycle within a predetermined period in order to collect the decision making information, and then provides the high-magnification photographing image (hair/scalp state information) jointly with received intermediate analysis result information for each cycle to generate the decision making information as provided reaction information (response and brainwave information to O and X through the input unit) according to each query,
collects, from the mobile terminal connected to a brainwave measurement device, brainwave change information according to a first query for decision making of the positive aspect (pros) according to the use of each recommended shampoo information/decision making of the negative aspect (cons) opposite thereto, brainwave change information according to a second query for decision making of the positive aspect (pros) according to the use of each scalp scaling information/decision making of the negative aspect (cons) opposite thereto, and brainwave change information according to a third query for decision making of the positive aspect (pros) according to the use of scalp massage information/decision making of the negative aspect (cons) opposite thereto, as brainwave information,
collects the decision making information as first parameter information which is decision making of a positive aspect (pros) depending on the use of each recommended shampoo information (the shampoo for the normal state, the shampoo for the dry state, the shampoo for the oily state, the shampoo for the sensitive state, the anti-dandruff shampoo, and the hair loss shampoo)/a negative aspect (cons) opposite thereto, second parameter information which is decision making of a positive aspect (pros) depending on the user of scalp scaling information/decision making of the negative aspect opposite thereto, and third parameter information which is decision making of a positive aspect (pros) depending on the use of scalp massage information/decision making of a negative aspect (cons) opposite thereto which are parameter information for the effect depending on the hair and scalp management behavior information,
collects the first to third parameter information by a scheme of providing the brainwave change information measured by the brainwave measurement device to the mobile terminal 100 as reference information, and receiving whether to use the component constituting each hair and scalp management behavior information from the mobile terminal for an intention expression (O and X) of the user through the input unit of the mobile terminal,
analyzes the brainwave change information provided to the mobile terminal by the transtheoretical model providing module by decision making of a negative aspect (cons) opposite thereto when a predetermined frequency or more increases in each initial reference brainwave information and a positive aspect (pros) when the predetermined frequency or more does not increase for a user who stares at the intermediate analysis result information for each cycle while the initial reference brainwave information is primarily classified into a delta wave, a theta wave, an alpha wave, a beta wave, and an gamma wave,
analyzes whether to maintain each normal state, whether the dry state is changed to the normal state, whether the oily state is changed to the normal state, whether the sensitive state is changed to the normal state, whether the dandruff scalp is changed to the normal state, and whether the hair loss state is changed to the normal state corresponding to the hair and scalp management behavior information, and the initial hair/scalp analysis information depending on each parameter information of the decision making information,
in a case where one of whether to maintain the normal state and whether each state is changed to the normal state received from the AI server 500 is positive and the decision making of the positive aspect (pros) and the decision making of the negative aspect (cons) opposite thereto are matched with each other in each parameter information of the decision making information for the hair and scalp management behavior information, analyzes the case as positive relevance to component information of the hair and scalp management behavior information jointly with the user unit information as the positive relevance information,
in a case where both whether to maintain the normal state and whether each state is changed to the normal state is negative and the decision making of the positive aspect (pros) and the decision making of the negative aspect (cons) opposite thereto are matched with each other in each parameter information of the decision making information for the hair and scalp management behavior information, analyzes the case as negative relevance to store component information of the hair and scalp management behavior information jointly with the user unit information as the negative relevance information,
analyzes relevance with hair and scalp management characteristics of the user and a transtheoretical model configuration factor,
collects component information {each recommended shampoo information (the shampoo for the normal state, the shampoo for the dry state, the shampoo for the oily state, the shampoo for the sensitive state, the anti-dandruff shampoo, and the hair loss shampoo), the scalp scaling information, and the scalp massage information} of each hair and scalp management behavior information corresponding to the positive relevance and the negative relevance collected on the database 430 for each user for each predetermined cycle to analyze a step indicating an intention for a behavior change of the transtheoretical model,
controls the transceiving unit to generate degree information for a change from a current hair and scalp state of the user to the normal state and generates the generated degree information as change process information, additionally receive decision making information and self efficacy information through the input unit of the mobile terminal for the user and generate step information for one of pre-plan, plan, preparation, behavior, and maintenance as a step showing an intention regarding the behavior change of the practice performer and transmit the generated step information to the mobile terminal through the network to control to output the degree information and the step information to the output unit of the mobile terminal 100, and
controls the transceiving unit to aggregate quantitative numerical values of the change process information, the decisional balance information, and the self efficacy information, and then generate step information (a quantity increases as a first range step proceeds to a fifth range step) corresponding to pre-plan when a range of the aggregated quantitative numerical value is a first range step, plan when the range of the aggregated quantitative numerical value is a second range step, preparation when the range of the aggregated quantitative numerical value is a third range step, behavior when the range of the aggregated quantitative numerical value is a fourth range step, and maintenance when the range of the aggregated quantitative numerical value is a fifth range step, and transmit the step information to the mobile terminal to control the step information to the output unit of the mobile terminal, and
a feedback providing module which generates a VR image according to the timeline for a 2D image for the high-magnification photographing image (hair/scalp state information) for generating the intermediate analysis result information for each cycle from an initially collected high-magnification photographing image (hair/scalp state information), and provides the generated VR image to the mobile terminal through the network,
in order to generate the VR image according to the cycle of each timeline, determines a plurality of focal positions and focuses for the high-magnification photographing image which is the 2D image, and computes a focal distance between respective focal positions for a plurality of focus 2D image data and computes a depth value which is in inverse proportion to the focal distance computed between the respective focal positions when a plurality of multiple focus 2D image data corresponding to the plurality of focus positions and focuses determined,
performs decoding of extracting and informationizing image information (a color, chroma, and brightness) corresponding to each pixel for a range of image data corresponding to each focus number of the high-magnification photographing image (hair/scalp state information) to generate decoded data and generates a polygon set to which a depth value computed for each pixel is reflected for a polygon which is a basic unit for expressing the decoded data as a 3D shape and then performs pixel mapping of attaching the decoded data onto the polygon set to generate each specified focus-specific virtual reality (VR) image, and
provides the set of the VR image according to the cycle of each timeline to the mobile terminal through the network to provide the scalp and hair state according to the hair and scalp management behavior information which is a behavior change process as the time elapses to the user as a realistic VR image through the HMD 100a connected to the mobile terminal through the short-range wireless communication.

19. The scalp and hair management system of claim 18, further comprising:
an information providing module providing, when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the decisional balance information to provide the decisional balance information in order to determine first to fifth range steps, approval item information extracted through extraction to the mobile terminal through the network jointly with the user ID with respect to response to the positive aspect in the approval item during moving to the higher step in information on five steps of pre-plan, plan, preparation, behavior, and maintenance as the change steps presented in the theoretical model by the initial information analysis module to know that a positive mind of the user causes the movement to the higher step,
providing, when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the decisional balance information to provide the decisional balance information in order to determine first to fifth range steps, non-approval item information extracted through extraction to the mobile terminal through the network jointly with the user ID with respect to response to the positive aspect in the non-approval item during moving to the lower step in information on five steps of pre-plan, plan, preparation, behavior, and maintenance as the change steps presented in the theoretical model by the initial information analysis module to know that a negative mind of the user causes the movement to the lower step,
when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, approval item information extracted through extraction to the mobile terminal through the network jointly with the user ID with respect to response to the positive aspect in the approval item during moving to the higher step in information on five steps of pre-plan, plan, preparation, behavior, and maintenance as the change steps presented in the theoretical model by the initial information analysis module to know that a positive mind of the user progresses the movement to the higher step,
when aggregating numerical values computed through a multiplication for a predetermined weight corresponding to a quantitative numerical value set for each step from the positive aspect to the negative aspect for each of the approval and non-approval items according to multiple selection for the positive aspect (very so, sometimes so, and so) and the negative aspect (not almost so and so not at all) like very so, sometimes so, so, not almost so, and so not at all for the self efficacy information to provide the self efficacy information, providing non-approval item information extracted through extraction to the mobile terminal through the network jointly with the user ID with respect to response to the positive aspect in the non-approval item during moving to the lower step in information on five steps of pre-plan, plan, preparation, behavior, and maintenance as the change steps presented in the theoretical model by the initial information analysis module to know that a positive mind of the user causes the movement to the lower step,
extracting, as first information, positive component information (when the decision making of the positive aspect is each parameter information of the decisional information which influences maintaining the normal state and changing to the normal state) of the hair and scalp management behavior information and negative component information (when the decision making of the negative aspect is each parameter information of the decisional information which influences maintaining the normal state and changing to the normal state) analyzed as the “positive relevance” stored in the database for each user ID,
extracting, as second information, positive component information (when the decision making of the negative aspect is each parameter information of the decisional information which influences not maintaining the normal state and not changing to the normal state) of the hair and scalp management behavior information and negative component information (when the decision making of the positive aspect is each parameter information of the decisional information which inversely influences not maintaining the normal state and not changing to the normal state) analyzed as the “negative relevance” stored in the database 430, and
generating graph information in which “positive element information” corresponding to positive component information of positive relevance which is first information and negative component information of negative relevance which is second information and “negative element information” corresponding to negative component information of positive relevance which is first information and positive component information of negative relevance which is second information during moving for each information for five steps of pre-plan, plan, preparation, behavior, and maintenance as the change steps presented in the theoretical model in the user unit information of each mobile terminal is represented as a timeline between neighboring steps, and providing approval item information (first information) extracted through extraction with respect to response to the positive aspect in the decisional balance information and the approval item for forming the decisional balance information during moving to the higher step in information on five steps of pre-plan, plan, preparation, behavior, and maintenance as the change steps presented in the theoretical model presented by the information providing module 424 and approval item information (second information) extracted through extraction with respect to response to the negative aspect in the decisional balance information and the approval items for forming the decisional balance information during moving to the lower step in information on five steps of pre-plan, plan, preparation, behavior, and maintenance as the change steps presented in the theoretical model to the mobile terminal 100 through the network for each user ID in real time.
